(19) United States
(12) Patent Application Publication
(10) Pub. No.: US 2010/0130476 A1
(43) Pub. Date: May 27, 2010

(54) COMPOUNDS THAT INDUCE PANCREATIC BETA-CELL EXPANSION

(75) Inventors: Weidong Wang, San Diego, CA (US); Peter G. Schultz, La Jolla, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Scripps Research Institute, La Jolla, CA (US)

(21) Appl. No.: 12/617,630

(22) Filed: Nov. 12, 2009

Related U.S. Application Data
(60) Provisional application No. 61/113,950, filed on Nov. 12, 2008.

(51) Int. Cl. A61K 31/519; C07D 409/14; C07D 409/04; C07D 413/14; C07D 473/16; C07D 403/04; C12N 5/00; A61K 31/5377; A61K 31/52; A61K 31/551; A61K 31/517; A61P 3/10

(52) U.S. Cl. 514/218; 544/295; 544/331; 544/122; 544/277; 540/575; 544/291; 435/375; 514/252.14; 514/275; 514/235.8; 514/263.4; 514/266.2

(57) ABSTRACT

The present invention relates to compounds and compositions for inducing the expansion of pancreatic β-cells. The invention further relates to a use of these expanded pancreatic β-cells to reversibly expand pancreatic β-cells and other quiescent cells to overcome deficits associated with degenerative and/or autoimmune diseases.

### CROSS-REFERENCES TO RELATED APPLICATIONS

This application is related to U.S. Provisional Application No. 61/113,950, filed Nov. 12, 2008, the disclosure of which is hereby incorporated by reference in its entirety for all purposes.

### STATEMENT AS TO RIGHTS TO INVENTIONS MADE UNDER FEDERALLY SPONSORED RESEARCH OR DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

Diabetes mellitus is a disease of the glucose regulatory system characterized by hyperglycemia (high glucose blood sugar). There are two main types of diabetes. Type 1 diabetes (also called juvenile onset or insulin-dependent diabetes) is due to an autoimmune attack on the insulin secreting pancreatic β-cells. Type 2 diabetes (also called adult onset or noninsulin-dependent diabetes) is characterized by a reduced mass of β-cells, reduced insulin secretion and resistance to the action of insulin (elevated concentrations of glucose in the blood).

Type 1 diabetes mellitus (T1DM) is characterized by a marked reduction in pancreatic β-cell mass (either by destruction or dysfunction) resulting in insufficient insulin secretion and, as a consequence, abnormally high blood glucose levels. Although daily insulin administration is used to treat hyperglycemia in T1DM patients, it does not fully recapitulate the strict control of blood glucose that is exerted by endogenous β-cells, and, as a result, does not prevent diabetic patients from eventually developing major damage to, for example, the kidneys, eyes, vascular and nervous systems. Transplantation of donor islet β-cells has been shown to successfully normalize blood glucose levels, but is limited by the scarcity of donor islets. Increasing evidence indicates that pancreatic β-cells replicate at a basal level in vivo, and can be stimulated to expand significantly to meet metabolic demand, for example, during pregnancy, obesity, or after partial pancreatectomy suggesting that external stimuli can be used to expand primary β-cells ex vivo for transplantation.

Clearly expansion of pancreatic β-cells is extremely beneficial, and hence compound to do the same would be of great utility. Such compound could provide for the ease and efficacy in the process of pancreatic β-cell expansion.

### SUMMARY OF THE INVENTION

The present invention relates to compounds and compositions for inducing the expansion of pancreatic β-cells and uses thereof to help control or prevent diseases associated with reduced β-cell functioning, such as diabetes.

The invention relates to compounds and compositions and methods of using the compounds to induce the expansion of pancreatic β-cells. In one embodiment, the present invention provides a compound of Formula Ia:

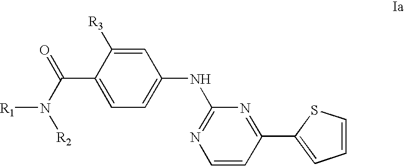

in which: R₁ is selected from hydrogen and C₁₋₆alkyl; R₂ is selected from C₁₋₆alkyl and —X₁NR₄R₅; wherein X₁ is C₁₋₄alkylene; R₄ and R₅ are independently selected from hydrogen and C₁₋₄alkyl; or R₄ and R₅ together with the nitrogen to which they are both attached, and optionally with another heteroatom chosen from the group O, S and N, form a 6 member heterocycle containing 1 to 2 heteroatoms; or R₁ and R₂ together with the nitrogen to which they are both attached, and optionally with another heteroatom chosen from the group O, S and N, form a 6 member heterocycle containing 1 to 2 heteroatoms; wherein the heterocycle formed from R₁ and R₂ or R₄ and R₅ can be optionally substituted with C₁₋₄alkyl; and R₃ is selected from hydrogen, halo, C₁₋₄alkyl, halo-substituted-C₁₋₄alkyl, C₁₋₄alkoxy and halo-substituted-C₁₋₄alkoxy; or the N-oxide derivatives, prodrug derivatives, protected derivatives, individual isomers and mixture of isomers thereof; or the pharmaceutically acceptable salts and solvates (e.g., hydrates) of such compounds.

In another embodiment, the present invention provides compounds of Formula Ib and Ic:

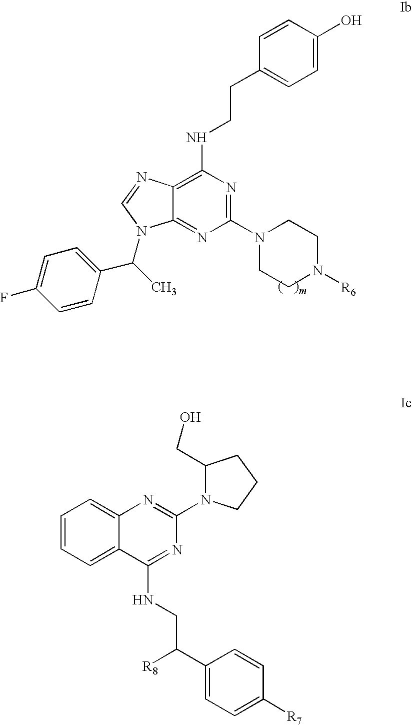

wherein: m is selected from 0, 1 and 2; and R₆ is C₁₋₆alkyl when the compound is of Formula Ib; or R₇ is selected from hydrogen, halo, C₁₋₄alkyl and C₁₋₄alkoxy; and R₈ is selected from hydrogen, C₁₋₄alkyl and C₆₋₁₀ aryl when the compound is of Formula Ic.

In another embodiment, the present invention provides a method of using a compound of Formula Id to increase the number of pancreatic β-cells; the method comprising contacting the pancreatic β-cells with a compound of Formula Id:

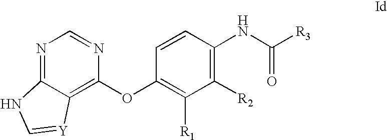

in which: Y is selected from N and CH; R₁ is selected from hydrogen and C₁₋₆alkyl; R₂ is selected from hydrogen and C₁₋₆alkyl; or R₁ and R₂, together with the fragment of the phenyl ring to which R₁ and R₂ are attached, and optionally with a heteroatom chosen from the group O, S, and N, form a C₆₋₁₀aryl or C₅₋₁₀heteroaryl; R₃ is selected from NR₄R₅ and X₁R₅, wherein X₁ is selected from a bond and C₁₋₄alkylene, R₄ is selected from hydrogen and C₁₋₆alkyl, and R₅ is selected from C₆₋₁₀aryl optionally substituted with 1 to 3 radicals independently selected from halo-substituted-C₁₋₆alkyl, C₁₋₆alkoxy, C₅₋₁₀heteroaryl, C₃₋₈heterocycloalkyl, C₅₋₁₀heteroaryl-C₁₋₄alkyl and C₃₋₈heterocycloalkyl-C₁₋₄alkyl; wherein the heteroaryl and heterocycloalkyl substituents of R₅ are optionally substituted with C₁₋₆alkyl; and the pharmaceutically acceptable salts, hydrates, solvates and isomers thereof.

In another embodiment, the present invention provides a method of using a compound of Formula Ie to increase the number of pancreatic β-cells, the method comprising contacting the pancreatic β-cells with a compound of Formula Ie:

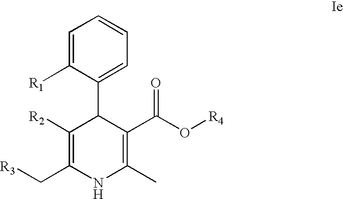

in which:
R₁ is selected from hydrogen, halo, C₁₋₄alkyl, C₁₋₄alkoxy, halo-substituted-C₁₋₄alkyl, halo-substituted-C₁₋₄alkoxy, phenoxy and benzyloxy; R₂ is selected from nitro and C(O)R₅, wherein R₅ is selected from hydrogen and C₁₋₄alkyl; R₃ is selected from hydrogen and C₁₋₄alkyl; or R₂ and R₃ together with the carbon atoms to which R₂ and R₃ are attached form a partially saturated 4 to 7 member cyclic group fused to the 1,4-dihydropyridine core of Formula Ie; and R₄ is selected from hydrogen and C₁₋₄alkyl.

In another embodiment of the invention, a method of treating a disease or disorder resulting from the reduced activity or destruction of pancreatic β-cells is provided the method comprising: administering to a patient in need of such treatment pancreatic β-cells expanded by a compound of the invention.

### BRIEF DESCRIPTION OF THE DRAWINGS

FIG. 1 illustrates that the novel Wnt agonists of the present invention are inducers of β-cell proliferation. (A) Chemical structures of 5-thiophene pyrimidines. (B) Dose-dependent effects of 1a on β-cell proliferation. Relative luminescence unit (RLU) was measured by CellTiter-Glo assay after 7-day incubation of growth-arrested R7T1 β cells with 1a, which was added at day 0 and refreshed at day 4; experiments were performed in quadruplicate. All data in this paper are presented as mean±SD, unless otherwise specified. (C) Dose-dependent effects of 1a on the activation of Super (8×) TOPFlash reporter. HEK293 cells transfected with Super (8×) TOPFlash reporter were treated with 1a at the indicated concentrations 24 h after transfection. Luciferase activity was measured 48 h after compound treatment. (D and E) The proliferative effect of 1a on rat primary β cells. (D) After incubation for 72 h with 2 μM 1a, replicating β cells were identified by double-immunofluorescence staining by using anti-C-peptide antibody to mark β cells (green) and antibody against Ki-67, a proliferation marker (red). Nuclear DNA was stained with DAPI (blue). (Scale bar, 10 μm.) (E) The percentage of Ki-67-positive cells of all C-peptide-positive cells, which corresponds to the fraction of replicating β cells. The number of proliferating primary β cells increased after incubation for 72 h in the presence of 2 μM 1a compared with DMSO control: 1a at 2 μM increases the replicating β cells ≈2-fold. (F) Compound 1a stimulates the translocation of β-catenin into the nucleus. Nuclear β-catenin accumulation (green arrows) was increased in 10 μM 1a-treated HEK293 cells; in DMSO-treated control cells, β-catenin staining is weak and appears to concentrate at the cell surface. (G) 1a-stimulated β-cell proliferation was abolished by the Wnt signaling antagonist Sulindac. R7T1 cells were treated with 10 μM 1a, 60 μM Sulindac, or 1 μM 2a as indicated in the figure. CellTiter-Glo activity was measured 7 days after compound treatment.

FIG. 2 illustrates that the LTCC agonists stimulate β-cell proliferation. (A) Chemical structures of dihydropyridine (DHP) derivatives. (B) Dose-dependent effect of 2a on β-cell proliferation. CellTiter-Glo assay was performed 7 days after incubation of growth-arrested R7T1 β cells with compounds; 0.1% of DMSO was used as control. Experiments were performed in quadruplicate. (C) LTCC agonists are responsible for the β-cell proliferation. Nimodipine (Nim), a known LTCC antagonist, resulted in growth-arrested R7T1 cell death. The proliferative effect of 2a (1 μM) on these β cells can be blocked by Nim in a dose-dependent fashion. At higher concentrations of Nim, β cells failed to survive even with 2a treatment; at lower concentrations of Nim, 2a-treated β cells proliferate. Each experiment was performed in quadruplicate. (D) Calcium influx is stimulated by 2a. Calcium influx was analyzed by using fluo-3 dye in MIN6 β cells. Fluo-3 dye was incubated for 1 h before compound treatment and measured by fluorescence laser imaging plate reader (FLIPR) for up to 5 min after compound treatment. (E and F) The proliferative effect of 2a on rat primary β cells. (E) After incubation for 72 h with 1 μM 2a, replicating β cells were identified by double-immunofluorescence staining by using anti-C-peptide antibody to mark β cells (green) and antibody against Ki-67, a proliferation marker (red). Nuclear DNA was stained with DAPI (blue). (Scale bar, 10 μm.) (F) The percentage of Ki-67-positive cells of all C-peptide-positive cells, which corresponds to the fraction of replicating β cells. The number of proliferating primary β cells increased after incubation for 72 h in the presence of 1 μM 2a compared with DMSO control: 2a at 1 μM increases the replicating β cells ≈2.5-fold. (G) 2a-induced (β-cell proliferation is reversible. R7T1 cells were able to proliferate for up to 3 weeks in the presence of 2a, but stopped proliferation upon its removal within 1 week. (H) The proliferative effect of 2a on human primary β cells. The number of proliferating human primary β cells increased ≈1.5-fold after incubation of 1 μM 2a for 72 h compared with DMSO as control. Replicating β cells were identified by double-immunofluorescence staining by using anti-C-peptide antibody to mark β cells (green) and antibody against Ki-67, a proliferation marker (red). Nuclear DNA was stained with DAPI (blue). (Scale bar, 10 μm.).

FIG. 3 illustrates the mechanistic studies of LTCC agonists. (A) Genes regulated by treatment with 2a (1 μM), the GLP-1 receptor agonist Exendin 4 (Ex-4, 5 nM), or both. Gene expression profiles were analyzed at 4 time points (30 min, 2 h, 6 h, and 24 h) after compound treatment of growth-arrested R7T1 β cells. Selected samples of differentially expressed genes are listed. Immediate-early response gene families, such as Fos, Jun, Egr, and Ier, were highly induced within 30 min after treatment with 2a. Irs2, a gene critical for β-cell survival and proliferation, was also up-regulated after treatment with 2a and Ex-4. Multiple entries for the same gene reflect multiple differentially expressed Affymetrix probe sets on the microarray. In addition, the expression of some RasGEFs was activated 2-6 h after treatment with 2a; this effect was enhanced by cotreating cells with Ex-4. Expression of Ccnd2 (cyclin D2) was up-regulated at 24 h after treatment with 2a; however, it too was up-regulated earlier (starting at 2 h posttreatment) and at higher levels after cotreatment with 2a and Ex-4. (B) The expression of several differentially expressed genes from microarray analysis was confirmed by real-time qRT-PCR. Growth-arrested R7T1 β cells were treated with 2a at the indicated time points; increases are relative to those found in DMSO-treated cells with each reaction performed in triplicate. (C) Activation of Ras signaling by 2a. Growth-arrested R7T1 β cells were treated with 2a at the indicated time points. Cell lysates were subjected to pull-down experiments by using the GST-Raf1 RBD fusion protein, followed by Western blot analysis to detect activated Ras (Ras-GTP, Upper); controls for Ras expression are also shown (total Ras, Lower).

FIG. 4 illustrates the additive effect of LTCC agonist 2a and the Glp-1 receptor agonist Ex-4. (A) Growth-arrested R7T1 β cells were incubated with DMSO, 2a (1 μM), Ex-4 (5 nM), or both for 7 days. Combined treatment with 2a and Ex-4 has an additive effect on the proliferation of growth-arrested R7T1 β cells as assessed by CellTiter-Glo (RLU). (B) HIT-T15 β cells were treated with DMSO, 2a (1 μM), Ex-4 (5 nM), or both 2a and Ex-4 for 1 day after serum-free culture for 24 h. Combined treatment with 2a and Ex-4 showed a higher proliferation rate as measured by BrdU incorporation. Values are presented as fold increase relative to DMSO-treated cells in serum-free medium; each experiment was performed in quadruplicate. (C) The proliferative effect of 2a and Ex-4 on human primary β cells. Human islets were cultured in the presence of DMSO, 2a (1 μM), Ex-4 (5 nM), or 2a and Ex-4 for 3 days. More proliferating human primary β cells were present when treated with both 2a and Ex-4. Replicating β cells were identified by double-immunofluorescence staining by using anti-C-peptide antibody to mark β cells (green) and antibody against Ki-67, a proliferation marker (red). Nuclear DNA was stained with DAPI (blue). (Scale bar, 10 μm.)

### DETAILED DESCRIPTION OF THE INVENTION

### Definitions

“Alkyl,” as a group and as a structural element of other groups, for example, halo-substituted-alkyl and alkoxy, can be either straight-chained or branched. C₁₋₄-alkoxy includes, methoxy, ethoxy, and the like. Halo-substituted alkyl includes trifluoromethyl, pentafluoroethyl, and the like.

“Aryl” means a monocyclic or fused bicyclic aromatic ring assembly containing six to ten ring carbon atoms. For example, aryl may be phenyl or naphthyl, preferably phenyl. “Arylene” means a divalent radical derived from an aryl group. “Heteroaryl” is as defined for aryl where one or more of the ring members are a heteroatom. For example, heteroaryl includes pyridyl, indolyl, indazolyl, quinoxalinyl, quinolinyl, benzofuranyl, benzopyranyl, benzothiopyranyl, benzo[1,3]dioxole, imidazolyl, benzo-imidazolyl, pyrimidinyl, furanyl, oxazolyl, isoxazolyl, triazolyl, tetrazolyl, pyrazolyl, thienyl, etc.

“Cycloalkyl” means a saturated or partially unsaturated, monocyclic, fused bicyclic or bridged polycyclic ring assembly containing the number of ring atoms indicated. For example, C₃₋₁₀cycloalkyl includes cyclopropyl, cyclobutyl, cyclopentyl, cyclohexyl, etc. “Heterocycloalkyl” means cycloalkyl, as defined in this application, provided that one or more of the ring carbons indicated, are replaced by a moiety selected from —O—, —N═, —NR—, —C(O)—, —S—, —S(O)— or —S(O)₂—, wherein R is hydrogen, C₁₋₄alkyl or a nitrogen protecting group. For example, C₃₋₈heterocycloalkyl as used in this application to describe compounds of the invention includes morpholino, pyrrolidinyl, piperazinyl, piperidinyl, piperidinylone, 2-Oxo-pyrrolidin-1-yl, 1,4-dioxa-8-aza-spiro[4.5]dec-8-yl, etc.

“Halogen” (or halo) represents bromo, chloro, fluoro or iodo.

“Treat,” “treating” and “treatment” refer to a method of alleviating or abating a disease and/or its attendant symptoms.

### DESCRIPTION OF THE PREFERRED EMBODIMENTS

The invention relates to compounds, compositions and methods of using the compounds to induce the expansion of pancreatic β-cells. In one embodiment, the present invention provides a compound of Formula Ia:

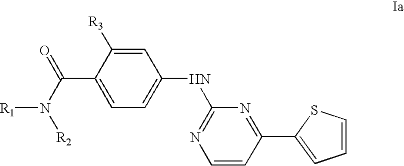

in which: R₁ is selected from hydrogen and C₁₋₆alkyl; R₂ is selected from C₁₋₆alkyl and —X₁NR₄R₅, wherein X₁ is C₁₋₄alkylene, R₄ and R₅ are independently selected from hydrogen and C₁₋₄alkyl; or R₄ and R₅ together with the nitrogen to which they are both attached, and optionally with another heteroatom chosen from the group O, S and N, form a 6 member heterocycle containing 1 to 2 heteroatoms; or R₁ and R₂ together with the nitrogen to which they are both attached, and optionally with another heteroatom chosen from the group O, S and N, form a 6 member heterocycle containing 1 to 2 heteroatoms; wherein the heterocycle formed from R₁ and R₂ or R₄ and R₅ can be optionally substituted with C₁₋₄alkyl. R₃, in Formula Ia, is selected from hydrogen, halo, C₁₋₄alkyl, halo-substituted-C₁₋₄alkyl, C₁₋₄alkoxy and halo-substituted-C₁₋₄alkoxy; or the N-oxide derivatives, prodrug derivatives, protected derivatives, individual isomers and mixture of isomers thereof; or the pharmaceutically acceptable salts and solvates (e.g., hydrates) of such compounds.

In one aspect, with reference to compounds of Formula Ia, R₁ is hydrogen; R₂ is selected from dimethyl-amino-ethyl, propyl and morpholino-ethyl; or R₁ and R₂ together with the nitrogen to which they are both attached form piperazinyl substituted with methyl.

In a further aspect are compounds selected from: (2-chloro-4-(4-(thiophen-2-yl)pyrimidin-2-ylamino)phenyl)(4-methylpiperazin-1-yl)methanone; (4-methylpiperazin-1-yl)(4-(4-(thiophen-2-yl)pyrimidin-2-ylamino)phenyl)methanone; 2-chloro-N-(2-(dimethylamino)ethyl)-4-(4-(thiophen-2-yl)pyrimidin-2-ylamino)benzamide; 2-chloro-N-propyl-4-(4-(thiophen-2-yl)pyrimidin-2-ylamino)benzamide; and 2-chloro-N-(2-morpholinoethyl)-4-(4-(thiophen-2-yl)pyrimidin-2-ylamino)benzamide.

In another embodiment, the present invention provides compounds selected from Formulas Ib and Ic:

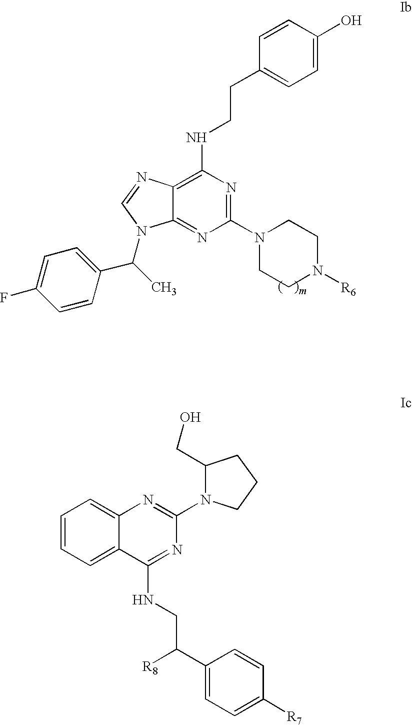

wherein: m is selected from 0, 1 and 2; and R₆ is C₁₋₆alkyl when the compound is of Formula Ib; or R₇ is selected from hydrogen, halo, C₁₋₄alkyl and C₁₋₄alkoxy; and R₈ is selected from hydrogen, C₁₋₄alkyl and C₆₋₁₀aryl when the compound is of Formula Ic.

In a further aspect, m is selected from 1 and 2; and R₆ is selected from methyl and ethyl when the compound is of Formula Ib; or R₇ is selected from hydrogen, bromo, methyl and methoxy; and R₈ is selected from hydrogen and phenyl when the compound is of Formula Ic.

In a further aspect are compounds selected from: 4-(2-(2-(4-ethylpiperazin-1-yl)-9-(1-(4-fluorophenyl)ethyl)-9H-purin-6-yl-amino)ethyl)phenol; 4-(2-(9-(1-(4-fluorophenyl)ethyl)-2-(4-methylpiperazin-1-yl)-9H-purin-6-ylamino)ethyl)phenol; 4-(2-(9-(1-(4-fluorophenyl)ethyl)-2-(4-methyl-1,4-diazepan-1-yl)-9H-purin-6-ylamino)ethyl)phenol; (1-(4-(4-methoxyphenethylamino)quinazolin-2-yl)pyrrolidin-2-yl)methanol; (1-(4-(4-bromophenethylamino)quinazolin-2-yl)pyrrolidin-2-yl)methanol; (1-(4-(4-methylphenethylamino)quinazolin-2-yl)pyrrolidin-2-yl)methanol; and (1-(4-(2,2-diphenylethylamino)quinazolin-2-yl)pyrrolidin-2-yl)methanol.

In another aspect is a method of using a compound of the invention to increase the number of pancreatic β-cells, the method comprising contacting the pancreatic β-cells with a compound of the invention (e.g., a compound of Formula Ia, Ib, Ic, etc.).

In a further aspect, the method is carried out in vivo, in vitro or ex vivo.

In a further aspect, the pancreatic β-cells are human pancreatic β-cells.

In another aspect is a method of treating a disease or disorder resulting from the reduced activity or destruction of pancreatic β-cells comprising administering to a patient in need of such treatment pancreatic β-cells expanded by a compound of the invention (e.g., a compound of Formula Ia, Ib, Ic, etc.).

In a further aspect, the disease or disorder is selected from type 1 and type 2 diabetes.

In another embodiment of the invention is a method of using a compound of Formula Id to increase the number of pancreatic (β-cells, the method comprising contacting the pancreatic β-cells with a compound of Formula Id:

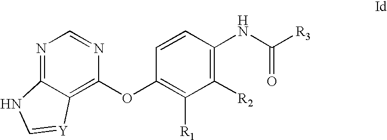

wherein: Y is selected from N and CH; R₁ is selected from hydrogen and C₁₋₆alkyl; R₂ is selected from hydrogen and C₁₋₆alkyl; or R₁ and R₂, together with the fragment of the phenyl ring to which R₁ and R₂ are attached, and optionally with a heteroatom chosen from the group O, S, and N, form a C₆₋₁₀aryl or C₅₋₁₀heteroaryl; and R₃ is selected from NR₄R₅ and X₁R₅, wherein X₁ is selected from a bond and C₁₋₄alkylene, R₄ is selected from hydrogen and C₁₋₆alkyl, and R₅ is selected from C₆₋₁₀ aryl optionally substituted with 1 to 3 radicals independently selected from halo-substituted-C₁₋₆ alkyl, C₁₋₆ alkoxy, C₅₋₁₀heteroaryl, C₃₋₈heterocycloalkyl, C₅₋₁₀heteroaryl-C₁₋₄alkyl and C₃₋₈heterocycloalkyl-C₁₋₄alkyl; wherein the heteroaryl and heterocycloalkyl substituents of R₅ are optionally substituted with C₁₋₆alkyl; and the pharmaceutically acceptable salts, hydrates, solvates and isomers thereof. Compounds of Formula Id are prepared according to PCT Publication No. WO2007/053343, the teachings of which are incorporated by reference.

In a further aspect, R₁ and R₂ are both hydrogen or R₁ and R₂, together with the fragment of the phenyl ring to which R₁ and R₂ are attached, and optionally with a heteroatom chosen from the group O, S, and N, form quinolinyl or naphthalenyl.

In a further aspect, R₃ is selected from NHR₅ and X₁R₅, wherein X₁ is selected from a bond and methylene; R₅ is selected from phenyl optionally substituted with 1 to 3 radicals independently selected from trifluoromethyl, methoxy, imidazolyl and piperazinyl-methyl; wherein the imidazolyl or piperazinyl substituents of R₅ are optionally substituted with methyl and ethyl.

In a further aspect are compounds of Formula Id selected from: 1-[4-(7H-Pyrrolo[2,3-d]pyrimidin-4-yloxy)-phenyl]-3-(3-trifluoromethyl-phenyl)-urea; 1-[4-(4-Ethyl-piperazin-1-ylmethyl)-3-trifluoromethyl-phenyl]-3-[4-(7H-pyrrolo[2,3-d]pyrimidin-4-yloxy)-phenyl]-urea; 1-[3-(4-Methyl-imidazol-1-yl)-5-trifluoromethyl-phenyl]-3-[4-(7H-pyrrolo[2,3-d]pyrimidin-4-yloxy)-phenyl]-urea; 1-(3,5-Dimethoxy-phenyl)-3-[4-(7H-pyrrolo[2,3-d]pyrimidin-4-yloxy)-phenyl]-urea; 1-[4-(9H-Purin-6-yloxy)-phenyl]-3-(3-trifluoromethyl-phenyl)-urea; 1-[3-(4-Methyl-imidazol-1-yl)-5-trifluoromethyl-phenyl]-3-[4-(9H-purin-6-yloxy)-phenyl]-urea; 1-[4-(4-Ethyl-piperazin-1-ylmethyl)-3-trifluoromethyl-phenyl]-3-[4-(9H-purin-6-yloxy)-phenyl]-urea; 1-[5-(7H-Pyrrolo[2,3-d]pyrimidin-4-yloxy)-quinolin-8-yl]-3-(3-trifluoromethyl-phenyl)-urea; N-[4-(7H-Pyrrolo[2,3-d]pyrimidin-4-yloxy)-phenyl]-2-(3-trifluoromethyl-phenyl)-acetamide; 2-[3-(4-Methyl-imidazol-1-yl)-5-trifluoromethyl-phenyl]-N-[4-(9H-purin-6-yloxy)-phenyl]-acetamide; N-[4-(7H-Pyrrolo[2,3-d]pyrimidin-4-yloxy)-phenyl]-3-trifluoromethyl-benzamide; 3-(4-Methyl-imidazol-1-yl)-N-[4-(7H-pyrrolo[2,3-d]pyrimidin-4-yloxy)-phenyl]-5-trifluoromethyl-benzamide; N-[4-(9H-Purin-6-yloxy)-phenyl]-3-trifluoromethyl-benzamide; 4-(4-Ethyl-piperazin-1-ylmethyl)-N-[4-(9H-purin-6-yloxy)-phenyl]-3-trifluoromethyl-benzamide; and N-[5-(9H-Purin-6-yloxy)-quinolin-8-yl]-3-trifluoromethyl-benzamide.

In a further aspect, the method is carried out in vivo, in vitro or ex vivo.

In a further aspect, the pancreatic β-cells are human pancreatic (β-cells.

In another embodiment of the invention is a method of treating a disease or disorder resulting from the reduced activity or destruction of pancreatic β-cells comprising administering to a patient in need of such treatment pancreatic β-cells expanded by a compound of the invention (including compounds of Formula Id of the invention).

In a further aspect, the disease or disorder is selected from type 1 and type 2 diabetes.

In another embodiment of the invention is a method of using a compound of Formula Ie to increase the number of pancreatic β-cells, the method comprising contacting the pancreatic β-cells with a compound of Formula Ie:

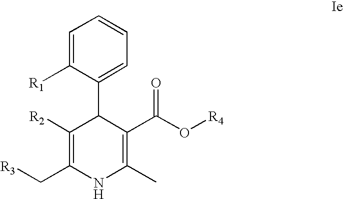

wherein: R₁ is selected from hydrogen, halo, C₁₋₄alkyl, C₁₋₄alkoxy, halo-substituted-C₁₋₄alkyl, halo-substituted-C₁₋₄alkoxy, phenoxy and benzyloxy; R₂ is selected from nitro and C(O)R₅, wherein R₅ is selected from hydrogen and C₁₋₄alkyl. R₃, in Formula Ie, is selected from hydrogen and C₁₋₄alkyl; or R₂ and R₃ together with the carbon atoms to which R₂ and R₃ are attached form a partially saturated 4 to 7 member cyclic group fused to the 1,4-dihydropyridine core of Formula Ie; and R₄ is selected from hydrogen and C₁₋₄alkyl.

In a further aspect, R₁ is selected from hydrogen, trifluoromethyl, propoxy, phenoxy, benzyloxy, butoxy and isopropoxy; R₂ is from nitro; R₃ is selected from hydrogen and C₁₋₄alkyl; or R₂ and R₃ together with the carbon atoms to which R₂ and R₃ are attached form a partially saturated 4 to 7 member cyclic group fused to the 1,4-dihydropyridine core of Formula Ie; and R₄ is selected from methyl, ethyl, propyl and isopropyl.

In a further aspect are compounds of Formula Ie selected from: (S)-methyl 2,6-dimethyl-5-nitro-4-(2-(trifluoromethyl)phenyl)-1,4-dihydropyridine-3-carboxylate; (S)-methyl 2,6-dimethyl-5-nitro-4-phenyl-1,4-dihydropyridine-3-carboxylate; (R)-methyl 2-methyl-5-oxo-4-(2-propoxyphenyl)-1,4,5,6,7,8-hexahydroquinoline-3-carboxylate; (R)-ethyl 2-methyl-5-oxo-4-(2-propoxyphenyl)-1,4,5,6,7,8-hexahydroquinoline-3-carboxylate; (R)-isopropyl 2-methyl-5-oxo-4-(2-propoxyphenyl)-1,4,5,6,7,8-hexahydroquinoline-3-carboxylate; (R)-isopropyl 4-(2-(benzyloxy)phenyl)-2-methyl-5-oxo-1,4,5,6,7,8-hexahydroquinoline-3-carboxylate; (R)-ethyl 4-(2-(benzyloxy)phenyl)-2-methyl-5-oxo-1,4,5,6,7,8-hexahydroquinoline-3-carboxylate; (R)-methyl 4-(2-(benzyloxy)phenyl)-2-methyl-5-oxo-1,4,5,6,7,8-hexahydroquinoline-3-carboxylate; (R)-methyl 4-(2-isopropoxyphenyl)-2-methyl-5-oxo-1,4,5,6,7,8-hexahydroquinoline-3-carboxylate; (R)-methyl 4-(2-butoxyphenyl)-2-methyl-5-oxo-1,4,5,6,7,8-hexahydroquinoline-3-carboxylate; (R)-isopropyl 4-(2-butoxyphenyl)-2-methyl-5-oxo-1,4,5,6,7,8-hexahydroquinoline-3-carboxylate; (R)-ethyl 4-(2-butoxyphenyl)-2-methyl-5-oxo-1,4,5,6,7,8-hexahydroquinoline-3-carboxylate; (R)-propyl 4-(2-butoxyphenyl)-2-methyl-5-oxo-1,4,5,6,7,8-hexahydroquinoline-3-carboxylate; and (R)-methyl 4-(2-butoxyphenyl)-2-methyl-5-oxo-1,4,5,6,7,8-hexahydroquinoline-3-carboxylate.

In a further aspect, the method is carried out in vivo, in vitro or ex vivo.

In a further aspect, the pancreatic β-cells are human pancreatic β-cells.

In another aspect is a method of treating a disease or disorder resulting from the reduced activity or destruction of pancreatic β-cells comprising administering to a patient in need of such treatment pancreatic β-cells expanded by a compound of the invention.

In a further aspect, the disease or disorder is selected from type 1 and type 2 diabetes.

In a further aspect, the method further comprises administering to the patient a GLP-1 receptor agonist, such as a GLP-1 analog.

### Methods/Uses for Inducing the Expanding if Pancreatic β-Cells

β-cells constitute the predominant type of cell in the islets of Langerhans of the pancreas. These pancreatic β-cells are particularly important because of their role manufacturing insulin to control blood glucose levels. New insulin-producing pancreatic β-cells are formed primarily by self-replication during adult life. Degeneration of the pancreatic β-cells is the main cause of type 1 (insulin-dependent) diabetes mellitus.

Type 1 diabetes mellitus (T1DM) is characterized by a marked reduction in pancreatic β-cell mass resulting in insufficient insulin secretion and, as a consequence, abnormally high blood glucose levels. Although daily insulin administration remains the most effective treatment for hyperglycemia in T1DM, it does not fully recapitulate the strict control of blood glucose that is exerted by endogenous β-cells, and, as a result, does not prevent diabetic patients from eventually developing major damage to the kidneys, eyes, vascular and nervous systems.

Increasing evidence indicates that pancreatic β-cells replicate at a basal level in vivo, and can be stimulated to expand significantly to meet metabolic demand, for example, during pregnancy, obesity, or after partial pancreatectomy. These observations suggest that it is possible to use external stimuli to expand primary β-cells ex vivo for transplantation, or even induce the regeneration of endogenous β-cell mass directly in the pancreas.

Transplantation of donor islet β-cells has been shown to successfully normalize blood glucose levels, but is limited by the scarcity of donor islets (β-cells from cadaver pancreases are in very short supply). This problem could be overcome by generating more β-cells from the available donors. Alternatively, β-cell mass in the pancreas could be induced to increase or regenerate in vivo.

The compounds and methods of the invention can be used to expand primary β-cells ex vivo for transplantation and/or can be used directly in diabetic patients to induce β-cell expansion to restore the regulation of glucose. A supply of β-cells to transplantation can be derived from the proliferation of existing β-cells, either isolated or within islets, by inducing differentiation of ES cells in β-cells, by inducing differentiation of isolated ductal epithelium into β-cells or islets, and by inducing transdifferentiation of related cell types, such as exocrine cells, hepatocytes or intestinal entereoendocrine cells into β-cells.

Provided are pancreatic β-cells expanded by a compound of the invention in combination with a therapeutic capable of enhancing the expansion of pancreatic β-cells in vivo, in vitro, or ex vivo (for example, a small molecule, an antibody, or the like) and optionally at least one pharmaceutically acceptable excipient or carrier. By a therapeutic capable of enhancing the expansion of pancreatic β-cells is meant: an activator of protein kinase C isozymes; an activator of the Hedgehog and Wnt signaling pathways; a selective GSK3-βinhibitor; and an agonist of the L-type calcium channels (LTCCs). In a preferred embodiment, pancreatic β-cells are expanded by a compound of the present invention in combination with an agonist of the glucagon-like peptide-1 (GLP-1) receptor. Examples of suitable GLP-1 receptor agonists include GLP-1 analogs, such as Exendin-4, exenatide (i.e., a synthetic version of Exendin-4), liraglutide, taspoglutide, etc. Other GLP-1 analogs know to and used by those of skill in the art (as well as those GLP-1 analogs to be developed) can be used in combination with the compounds of the present invention, such as the compounds of Formulae Ia-Ie and, in particular, the compounds of Formula Ie, to induce the expansion of pancreatic β-cells. Those of skill in the art will appreciate that the GLP-1 receptor agonist, such as a GLP-1 analog, can be administered in combination with a compound of the present invention or, alternatively, it can be administered prior to or after administration of a compound of the present invention.

By pharmaceutically acceptable is meant a material that is not biologically or otherwise undesirable, i.e., the material may be administered to a subject or cell, without causing undesirable biological effects or interacting in a deleterious manner with the other components of the pharmaceutical composition in which it is contained. The carrier or excipient is selected to minimize degradation of the active ingredient and to minimize adverse side effects in the subject or cell.

The compositions are formulated in any conventional manner for use in the methods described herein. Administration is via any route known to be effective by one of ordinary skill. For example, the compositions is administered orally, parenterally (e.g., intravenously), by intramuscular injection, by intraperitoneal injection, transdermally, extracorporeally, intranasally or topically.

For oral administration, the compositions take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets are coated by methods well known in the art. Liquid preparations for oral administration take the form of, for example, solutions, syrups or suspensions, or they may be presented as a dry product for constitution with water or other suitable vehicle before use. Such liquid preparations are prepared by conventional means with pharmaceutically acceptable additives such as suspending agents (e.g., sorbitol syrup, cellulose derivatives or hydrogenated edible fats); emulsifying agents (e.g., lecithin or acacia); nonaqueous vehicles (e.g., almond oil, oily esters, ethyl alcohol or fractionated vegetable oils); and preservatives (e.g., methyl or propyl-p-hydroxybenzoates or sorbic acid). The preparations optionally contain buffer salts, flavoring, coloring and sweetening agents as appropriate.

The compositions are formulated for parenteral administration by injection, e.g., by bolus injection or continuous infusion. Formulations for injection are presented in unit dosage form, e.g., in ampules or in multi-dose containers, with or without an added preservative. The compositions take such forms as suspensions, solutions or emulsions in oily or aqueous vehicles, and may contain formulatory agents such as suspending, stabilizing and/or dispersing agents. Alternatively, the active ingredient is in powder form for constitution with a suitable vehicle, e.g., sterile pyrogen-free water, before use. In general, water, suitable oil, saline, aqueous dextrose (glucose), and related sugar solutions and glycols such as propylene glycol or polyethylene glycols are suitable carriers for parenteral solutions. Solutions for parenteral administration contain, for example, a water soluble salt of the active ingredient, suitable stabilizing agents and, if necessary, buffer substances. Antioxidizing agents such as sodium bisulfate, sodium sulfite or ascorbic acid, either alone or combined, are suitable stabilizing agents. Also citric acid and its salts and sodium ethylenediaminetetraacetic acid (EDTA) are optionally used. In addition, parenteral solutions optionally contain preservatives such as benzalkonium chloride, methyl- or propyl-paraben and chlorobutanol. Suitable pharmaceutical carriers are described in Remington: The Science and Practice of Pharmacy, 21st Edition, David B. Troy, ed., Lippicott Williams & Wilkins (2005), which is incorporated by reference in its entirety at least for the material related to pharmaceutical carriers and compositions.

The compositions are optionally formulated as a depot preparation. Such long acting formulations are optionally administered by implantation. Thus, for example, the compositions are formulated with suitable polymeric or hydrophobic materials (for example as an emulsion in an acceptable oil) or ion exchange resins, or as sparingly soluble derivatives, for example, as a sparingly soluble salt. The compositions are applied to or embedded with implants concurrent with or after surgical implant.

Additionally, standard pharmaceutical methods are employed to control the duration of action. These include control release preparations and appropriate macromolecules, for example, polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation are adjusted in order to control release. Optionally, the agent is incorporated into particles of polymeric materials such as polyesters, polyamino acids, hydrogels, poly (lactic acid) or ethylenevinylacetate copolymers. In addition to being incorporated, these agents are optionally used to trap the compound in microcapsules.

A composition for use in the methods described herein is optionally formulated as a sustained and/or timed release formulation. Such sustained and/or timed release formulations are made by sustained release means or delivery devices that are well known to those of ordinary skill in the art. The compositions are used to provide slow or sustained release of one or more of the active ingredients using, for example, hydropropylmethyl cellulose, other polymer matrices, gels, permeable membranes, osmotic systems, multilayer coatings, microparticles, liposomes, microspheres or a combination thereof to provide the desired release profile in varying proportions. Suitable sustained release formulations are selected for use with the compositions described herein. Thus, single unit dosage forms suitable for oral administration, such as, but not limited to, tablets, capsules, gelcaps, caplets, powders, that are adapted for sustained release are used.

The compositions are optionally delivered by a controlled-release system. For example, the composition is administered using intravenous infusion, an implantable osmotic pump, liposomes, or other modes of administration. A controlled release system is placed in proximity to the target.

Optionally, it is desirable to administer the composition locally, i.e., to the area in need of treatment. For example, the composition is administered by injection into the bone marrow of a long bone, for example. Local administration is achieved, for example, by local infusion during surgery, topical application (e.g., in conjunction with a wound dressing after surgery), injection, catheter, suppository, or implant. An implant is of a porous, nonporous, or gelatinous material, including membranes, such as sialastic membranes, or fibers.

The pharmaceutical compositions described herein are administered by any conventional means available for use in conjunction with pharmaceuticals, either as individual therapeutic active ingredients or in a combination of therapeutic active ingredients. They are optionally administered alone, but are generally administered with a pharmaceutical carrier selected on the basis of the chosen route of administration and standard pharmaceutical practice.

The compounds described herein are provided in a pharmaceutically acceptable form including pharmaceutically acceptable salts and derivatives thereof. The term pharmaceutically acceptable form refers to compositions including the compounds described herein that are generally safe, relatively nontoxic and neither biologically nor otherwise undesirable. These compositions optionally include pharmaceutically acceptable carriers or stabilizers that are nontoxic to the cell or subject being exposed thereto at the dosages and concentrations employed. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (Uniqema, United Kingdom), polyethylene glycol (PEG), and PLURONICS™ (BASF, Germany).

The term pharmaceutically acceptable acid salts and derivatives refers to salts and derivatives of the compounds of Formula Ia-Ie described herein that retain the biological effectiveness and properties as described, and that are not biologically or otherwise undesirable. Pharmaceutically acceptable salts are formed, for example, with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

The chemical stability of a composition comprising a compound of Formula I (i.e., a compound of Formula Ia-Ie) or a pharmaceutically acceptable salt or ester thereof is enhanced by methods known to those of skill in the art. For example, an alkanoic acid ester of a polyethoxylated sorbitol (a polysorbate) is added to a composition containing a compound of Formula I in an amount effective to enhance the chemical stability of the compound.

The data obtained from the cell culture assays and animal studies are optionally used in formulating a range of dosage for use in humans. The dosage of such compounds lies preferably within a range of circulating concentrations that include little or no toxicity. The dosage varies within this range depending upon the dosage form employed and the route of administration utilized. For any compound used in the provided methods, the therapeutically effective dose is estimated initially from cell culture assays.

Also provided herein is a pack or kit comprising one or more containers filled with one or more of the ingredients described herein. Such kits optionally comprise solutions and buffers as needed or desired. The kit optionally includes an expanded population of stem cells made by the methods described above or can contain containers or compositions for making an expanded population of pancreatic β-cells. Optionally associated with such pack(s) or kit(s) are instructions for use.

Also provided is a kit for providing an effective amount of a compound of the invention to increase pancreatic β-cells in a subject comprising one or more doses of the compound for use over a period of time, wherein the total number of doses of the compound of the invention in the kit equals the effective amount sufficient to increase pancreatic β-cells in a subject. The period of time is from about one to several days or weeks or months. Thus, the period of time is from at least about 5, 6, 7, 8, 10, 12, 14, 20, 21, 30 or 60 days or more or any number of days between one and 90.

### Processes for Making Compounds of the Invention

The present invention also includes processes for the preparation of compounds of the invention. In the reactions described in the examples, infra, it can be necessary to protect reactive functional groups, for example hydroxy, amino, imino, thio or carboxy groups, where these are desired in the final product, to avoid their unwanted participation in the reactions. Conventional protecting groups can be used in accordance with standard practice, for example, see T. W. Greene and P. G. M. Wuts in “Protective Groups in Organic Chemistry”, John Wiley and Sons, 1991.

### Additional Processes for Making Compounds of the Invention

A compound of the invention can be prepared as a pharmaceutically acceptable acid addition salt by reacting the free base form of the compound with a pharmaceutically acceptable inorganic or organic acid. Alternatively, a pharmaceutically acceptable base addition salt of a compound of the invention can be prepared by reacting the free acid form of the compound with a pharmaceutically acceptable inorganic or organic base. Alternatively, the salt forms of the compounds of the invention can be prepared using salts of the starting materials or intermediates.

The free acid or free base forms of the compounds of the invention can be prepared from the corresponding base addition salt or acid addition salt from, respectively. For example, a compound of the invention in an acid addition salt form can be converted to the corresponding free base by treating with a suitable base (e.g., ammonium hydroxide solution, sodium hydroxide, and the like). A compound of the invention in a base addition salt form can be converted to the corresponding free acid by treating with a suitable acid (e.g., hydrochloric acid, etc.).

Compounds of the invention in unoxidized form can be prepared from N-oxides of compounds of the invention by treating with a reducing agent (e.g., sulfur, sulfur dioxide, triphenyl phosphine, lithium borohydride, sodium borohydride, phosphorus trichloride, tribromide, or the like) in a suitable inert organic solvent (e.g. acetonitrile, ethanol, aqueous dioxane, or the like) at 0 to 80° C.

Prodrug derivatives of the compounds of the invention can be prepared by methods known to those of ordinary skill in the art (e.g., for further details see Saulnier et al., (1994), Bioorganic and Medicinal Chemistry Letters, Vol. 4, p. 1985). For example, appropriate prodrugs can be prepared by reacting a nonderivatized compound of the invention with a suitable carbamylating agent (e.g., 1,1-acyloxyalkylcarbanochloridate, para-nitrophenyl carbonate, or the like).

Protected derivatives of the compounds of the invention can be made by means known to those of ordinary skill in the art. A detailed description of techniques applicable to the creation of protecting groups and their removal can be found in T. W. Greene, “Protecting Groups in Organic Chemistry”, 3^{rd} edition, John Wiley and Sons, Inc., 1999.

Compounds of the present invention can be conveniently prepared, or formed during the process of the invention, as solvates (e.g., hydrates). Hydrates of compounds of the present invention can be conveniently prepared by recrystallization from an aqueous/organic solvent mixture, using organic solvents such as dioxin, tetrahydrofuran or methanol.

Compounds of the invention can be prepared as their individual stereoisomers by reacting a racemic mixture of the compound with an optically active resolving agent to form a pair of diastereoisomeric compounds, separating the diastereomers and recovering the optically pure enantiomers. While resolution of enantiomers can be carried out using covalent diastereomeric derivatives of the compounds of the invention, dissociable complexes are preferred (e.g., crystalline diastereomeric salts). Diastereomers have distinct physical properties (e.g., melting points, boiling points, solubilities, reactivity, etc.) and can be readily separated by taking advantage of these dissimilarities. The diastereomers can be separated by chromatography, or preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen, “Enantiomers, Racemates and Resolutions”, John Wiley And Sons, Inc., 1981.

In summary, the compounds of Formula I can be made by a process, which involves:

(a) optionally converting a compound of the invention into a pharmaceutically acceptable salt;

(b) optionally converting a salt form of a compound of the invention to a nonsalt form;

(c) optionally converting an unoxidized form of a compound of the invention into a pharmaceutically acceptable N-oxide;

(d) optionally converting an N-oxide form of a compound of the invention to its unoxidized form;

(e) optionally resolving an individual isomer of a compound of the invention from a mixture of isomers;

(f) optionally converting a nonderivatized compound of the invention into a pharmaceutically acceptable prodrug derivative; and

(g) optionally converting a prodrug derivative of a compound of the invention to its nonderivatized form.

Insofar as the production of the starting materials is not particularly described, the compounds are known or can be prepared analogously to methods known in the art or as disclosed in the Examples hereinafter.

One of skill in the art will appreciate that the above transformations are only representative of methods for preparation of the compounds of the present invention, and that other well known methods can similarly be used.

### EXAMPLES

The present invention is further exemplified, but not limited, by the following examples that illustrate the preparation of compounds of Formula I (Examples) according to the invention.

### Example 1

### a) (2-chloro-4-(4-(thiophen-2-yl)pyrimidin-2-ylamino)phenyl)(4-methylpiperazin-1-yl)methanone

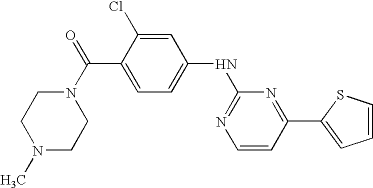

The reaction mixture of 2,4-dichloropyrimidine (100 mg, 0.67 mmol) and thiophen-2-ylboronic acid (85 mg, 0.67 mmol) in dioxane (10 mL) was treated with Cs₂CO₃ (1.0 g, 3.06 mmol), and Pd(Ph₃P)₄ (30 mg, 0.026 mmol). The reaction mixture was heated at 100° C. overnight under an argon atmosphere. The reaction was extracted with EtOAc and H₂O. The crude compound was purified by column chromatography (Hexane 100% to hexane/CH₂Cl₂=1:1) to afford an orange color solid (2-chloro-4-(thiophen-2-yl)pyrimidine) LC-MS: calcd M⁺ for C₈H₅ClN₂S 196; found 197 (M+H). The reaction mixture of 2-chloro-4-(thiophen-2-yl)pyrimidine (30 mg, 0.15 mmol) and 4-amino-2-chlorobenzoic acid (26 mg, 0.15 mmol) in dioxane (3 mL) was treated with Pd₂ dba₃ (45 mg, 0.05 mmol), Xantphos (87 mg, 0.15 mmol), and Cs₂CO₃ (488 mg, 1.5 mmol) and was heated at 100° C. overnight under an argon atmosphere. Crude compound was purified by HPLC to afford yellow solid (2-chloro-4-(4-(thiophen-2-yl)pyrimidin-2-ylamino)benzoic acid) LC-MS: calcd M⁺ for C₁₅H₁₀ClN₃O₂S 331; found 332 (M+H). The reaction mixture of 2-chloro-4-(4-(thiophen-2-yl)pyrimidin-2-ylamino)benzoic acid (100 mg, 0.302 mmol) and 1-methylpiperazine (36 mg, 0.36 mmol) in the MC/DMF=5:1 (6 mL) was treated with DIEA (46.6 mg, 0.36 mmol) and HATU (136 mg, 0.36 mmol). The reaction mixture was stirred at room temperature overnight. Crude compound was purified by HPLC to afford yellow compound. LC-MS: calcd M⁺ for C₂₀H₂₀ClN₅OS 413.1; found, 414.1 (M+H).

By repeating the procedure described in the above example, using appropriate starting materials, the following compounds of Formula Ia, as identified in Table 1, are obtained.

TABLE 1

| | | Physical Data |
|---|---|---|
| | | ¹H NMR |
| Example | | 400 MHz (CDCl₃) |
| Number | Structure | and/or MS (m/z) |
| 2 | 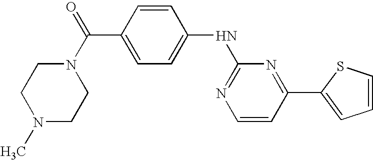 | |
| 3 | 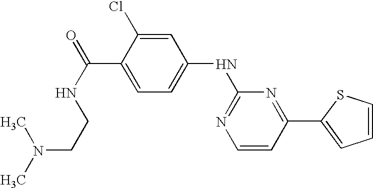 | |
| 4 | 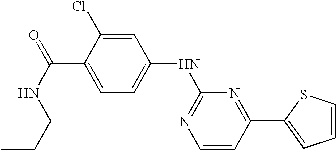 | |
| 5 | 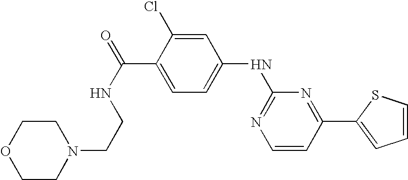 | |
| 5A | 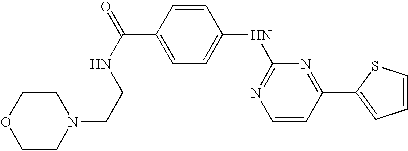 | |

### Example 2

### b) 4-(2-(2-(4-ethylpiperazin-1-yl)-9-(1-(4-fluorophenyl)ethyl)-9H-purin-6-yl-amino)ethyl)phenol

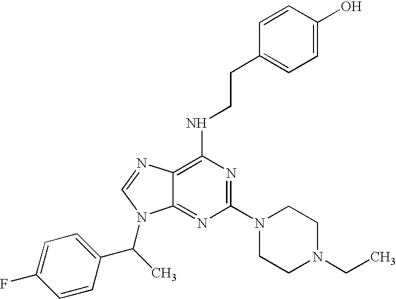

The reaction mixture of 2,6-dichloro-9H-purine (500 mg, 2.65 mmol) and 1-(4-fluorophenyl)ethanol (810 mg, 5.3 mmol) in anhydrous THF (40 mL) was treated with triphenylphosphine (1.38 g, 5.3 mmol). The reaction mixture was cooled down to 0° C. Diethyl azodicarboxylate (DEAD) (591.6 mg, 3.4 mmol) was slowly added (drop wise) to the reaction mixture. The reaction mixture was stirred continuously at 0° C. for several hours and stirred at room temperature overnight. Crude compound was purified by column chromatography to afford a light yellow oil (2,6-dichloro-9-(1-(4-fluorophenyl)ethyl)-9H-purine). The compound was used in the next step without further purification. The reaction mixture of 2,6-dichloro-9-(1-(4-fluorophenyl)ethyl)-9H-purine (67.0 mg, 0.21 mmol) and 4-(2-aminoethyl)phenol (28.7 mg, 0.21 mmol) in n-BuOH (2 mL) was treated with DIEA (54 mg, 0.42 mmol). The reaction mixture was heated at 80° C. for 4 hours. The reaction mixture was extracted with dichloromethane and H₂O. Crude compound was purified by column chromatography (Hexane 100% to CH₂Cl₂/MeOH=20:1) to afford a yellow oil (4-(2-(2-chloro-9-(1-(4-fluorophenyl)ethyl)-9H-purin-6-ylamino)ethyl)phenol). The reaction mixture of 4-(2-(2-chloro-9-(1-(4-fluorophenyl)ethyl)-9H-purin-6-ylamino)ethyl)phenol (127.1 mg, 0.31 mmol), DIEA (80.0 mg, 0.62 mmol), and 1-ethylpiperazine (70 mg, 0.62 mmol) in n-BuOH (4 mL) was heated at 120° C. overnight. Crude compound was purified by HPLC to afford a light yellow compound (4-(2-(2-(4-ethylpiperazin-1-yl)-9-(1-(4-fluorophenyl)ethyl)-9H-purin-6-ylamino)ethyl)phenol). LC-NMR (CD₃OD): 1.3 ppm (3H, m), 2.0 ppm (3H, d), 2.58 ppm (2H, m), 2.6 ppm (4H, m), 2.9 ppm (2H, m), 3.8 ppm (2H, m), 3.9 ppm (4H, m), 5.8 ppm (1H, dd), 6.8 ppm (2H, d), 7.18 ppm (4H, m), 7.4 ppm (2H, m), 7.82 ppm (1H, S). MS: calcd M+ for C₂₇H₃₂FN₇O 489.2; found 490.3 (M+H).

By repeating the procedure described in the above example, using appropriate starting materials, the following compounds of Formula Ib, as identified in Table 2, are obtained.

TABLE 2

| | | Physical Data |
|---|---|---|
| | | ¹H NMR |
| Example | | 400 MHz (CDCl₃) |
| Number | Structure | and/or MS (m/z) |
| 7 | 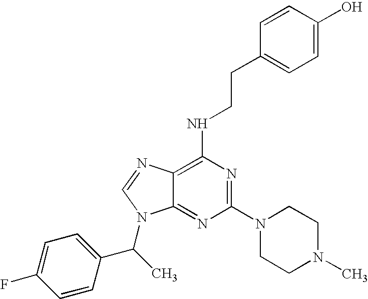 | LCMS calcd M+ for C26H30FN7O 475.2; Found 476.3 (M + H) |
| 8 | 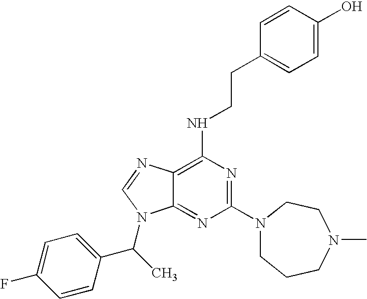 | LCMS calcd M+ for C27H32FN7O 489.2 Found: 490.3 (M + H) |

### Example 3

### c) (1-(4-(4-methoxyphenethylamino)quinazolin-2-yl)pyrrolidin-2-yl)methanol

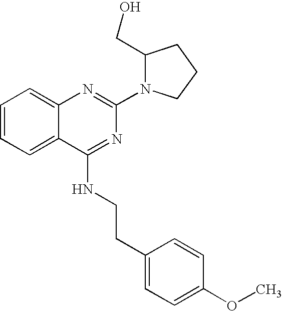

The reaction mixture of quinazoline-2,4(1H,3H)-dione (1 g) and dimethylaniline (0.5 mL) in POCl₃ (5 mL) was heated overnight. The reaction mixture was poured into ice. The precipitant was filtered and collected resulting in a grey color solid (2,4-dichloroquinazoline). The reaction mixture of 2,4-dichloroquinazoline (300 mg, 1.5 mmol) and 2-(4-methoxyphenyl)ethanamine (226 mg, 1.5 mmol) in n-BuOH (3 mL) was treated with DIEA (excess) and heated at 80° C. overnight. Crude compound was purified by column chromatography (Hexane 100% to CH₂Cl₂/MeOH=15:1) to afford a yellow oil (2-chloro-N-(4-methoxyphenethyl)quinazolin-4-amine). The reaction mixture of 2-chloro-N-(4-methoxyphenethyl)quinazolin-4-amine (100 mg, 0.32 mmol) and L-prolinol (32.3 mg, 0.32 mmol) in n-BuOH (1 mL) was treated with DIEA (81.27 mg, 0.63 mmol) and heated at 120° C. overnight. Crude compound was purified by HPLC to afford compound ((1-(4-(4-methoxyphenethylamino)quinazolin-2-yl)pyrrolidin-2-yl)methanol). NMR (CDCl₃): 1.78 ppm (1H, m), 1.98 ppm (1H, m), 2.00 pm (1H, m), 2.40 ppm (2H, m), 3.05 ppm (2H, t), 3.75 ppm (2H, m), 3.85 ppm (5H, m), 4.05 ppm (1H, m), 4.45 ppm (1H, m), 6.0 ppm (1H, br S), 6.94 ppm (2H, d), 7.05 ppm (1H, t), 7.2 ppm (2H, d), 7.45 ppm (2H, t), 7.55 pm (1H, t). LC-MS: calcd M+ for C22H26N4O2 378.2; found 379.2.

By repeating the procedure described in the above example, using appropriate starting materials, the following compounds of Formula Ic, as identified in Table 3, are obtained.

TABLE 3

| | | Physical Data |
|---|---|---|
| | | ¹H NMR |
| Example | | 400 MHz (CDCl₃) |
| Number | Structure | and/or MS (m/z) |
| 10 | 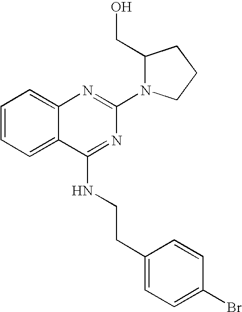 | LCMS: Calcd M+ for C21H23BrN4O: 426.1 Found (M + H): 427.1 |
| 11 | 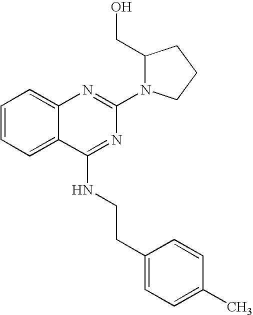 | LCMS: Calcd M+ for C22H26N4O: 362.2 Found (M + H): 363.2 |
| 12 | 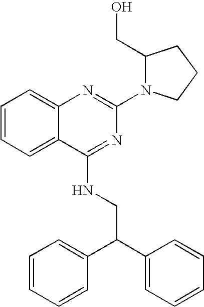 | LCMS: Calcd M+ for C27H28N4O: 424.2 Found (M + H): 425.3 |

### Example 4

### Assays

The following assays are used to assess the activity of the compounds of the invention to facilitate the expansion of pancreatic β-cells.

### Cell Culture

R7T1 β-cells are expanded in growth medium (DMEM with 15% horse serum and 2.5% FBS) in the presence of 10 μg/ml doxycycline. (3TC-tet cells are expanded in growth medium (DMEM with 15% horse serum and 2.5% FBS) in the absence of 10 μg/ml doxycycline. Hamster HIT-T15 β-cells are grown in F12 medium with 15% horse serum and 2.5% FBS. MIN6 cells are cultured in DMEM with 15% FBS. HEK293 cells are grown according to instructions from ATCC (http://www.atcc.org/).

### High-Throughput Screen

R7T1 β-cells are expanded in large quantities in growth medium in the presence of 10 μg/ml doxycycline and then are growth arrested by removal of doxycycline for one week. For the primary screen, growth-arrested cells are plated onto 384- or 1536-well plates in growth medium without doxycycline at a density of 4000 cells/well in 384-well plate or 1000 cells/well in 1536-well plate in an automated high-throughput screening platform (designated as Day 0). A heterocycle library of 850,000 compounds (at a final concentration of 1.25 μM) is added at Day 1 and refreshed at Day 5, and the effects on β-cell replication are determined at Day 8 using the CellTiter Glo assay system (a luminescent assay measuring intracellular ATP content which is directly proportional to cell number). For data analysis, the median of a plate is calculated and the activity of each well is the signal/median of the plate. A signal in any well over 1.5 of the median is regarded as a primary hit. For the reconfirmation screen, growth-arrested R7T1 cells are plated onto 384-well plates and screened against all primary hits in quadruplicate. To eliminate tetracycline mimetics among all reconfirmed primary hits, βTC-tet cells are plated onto 384-well plates at a density of 2000 cells/well in growth medium in the presence of 10 μg/ml doxycycline, and 1.25 μM compound is added after overnight incubation and refreshed 4 days later. The effects on β-cell replication are then determined at Day 8 using the CellTiter Glo assay system. Any compound that increases the readings more than 1.5 fold is regarded as a hit.

Compounds of the invention expand the pancreatic β-cell population by about 2.5 to about 5 fold. For example, (2-chloro-4-(4-(thiophen-2-yl)pyrimidin-2-ylamino)phenyl)(4-methylpiperazin-1-yl)methanone (Example 1) expanded the pancreatic β-cell population by 2.5 fold; 4-(2-(2-(4-ethylpiperazin-1-yl)-9-(1-(4-fluorophenyl)ethyl)-9H-purin-6-yl-amino)ethyl)phenol (Example 6) expanded the pancreatic β-cells by between 4 and 5 fold; and (1-(4-(4-methoxyphenethylamino)quinazolin-2-yl)pyrrolidin-2-yl)methanol (Example 9) expanded the pancreatic β-cells by 3 fold.

### Rat or Human Islet Preparations

Islets are isolated by the standard Liberase digestion method from the pancreata of adult Sprague-Dawley rats (200-250 g) (Liberase III; Roche Applied Science) and cultured in RPMI medium with 10% FBS. Briefly, 6 ml of ice-cold Liberase solution (Roche Applied Science) is injected into the pancreas via the common bile duct. After dissection, the pancreas is incubated for 35 minutes at 37° C. and then further dissociated by repeated pipetting using a 10-ml pipette. Islets are purified by Histopaque-1.077 (Sigma) density gradient centrifugation and are manually picked using a stereomicroscope. Islets are allowed to recover from the isolation procedure by culture overnight in RPMI medium, 10% FBS using plastic dishes to which they do not attach. Human islets are obtained through the Juvenile Diabetes Research Foundation Islet Distribution Program by Islet Cell Resource Center in Minnesota, USA. The purity and viability of human islets are reported to be 30-70% and 50-70%, respectively. Human islets are cultured in CMRL medium containing 10% FBS.

### In Vitro β-Cell Proliferation Assay

Primary β-cell proliferation assay: freshly isolated rat or human islets or trypsinized single cell suspensions of rat islets are cultured in vitro either in the presence or absence of compound for 72 hours. Islets or single suspension cells are fixed with 4% formalin solution (Sigma) and stained by standard immuno-fluorescence techniques for C-peptide (anti-human C-peptide antibody from Raybiotech, Inc., and anti-rat C-peptide antibody from Linco, Inc.) and Ki-67 (anti-Ki-67 antibody from Abcam), a marker of proliferating cells. Nuclear DNA is stained with DAPI (Molecular Probes). At least 30,000 C-peptide positive cells are counted in each experiment, performed in triplicate. The images are taken on a Nikon Eclipse TE300 microscope with 200× magnification. Proliferating C-peptide/Ki-67 double positive β-cells are counted manually.

Compounds of the invention increase the C-peptide/Ki double positive β-cells by at least 2-fold in rat and at least 1.5-fold in human. For example: (2-chloro-4-(4-(thiophen-2-yl)pyrimidin-2-ylamino)phenyl)(4-methylpiperazin-1-yl)methanone (Example 1) results in a 2 fold increase in rat C-peptide/Ki-67 double positive β-cells; and (S)-methyl 2,6-dimethyl-5-nitro-4-(2-(trifluoromethyl)phenyl)-1,4-dihydropyridine-3-carboxylate results in a 2.5 fold increase in rat C-peptide/Ki-67 double positive β-cells, and 1.5 fold increase in human C-peptide/Ki-67 double positive β-cells.

BrdU incorporation assay: HIT-T15 or MIN6 (β-cell proliferation is assessed by 5-bromo-2-deoxyuridine (BrdU) incorporation ELISA (Roche). Cells are seeded on 96-well plates at 5×10³ cells/well in serum-containing medium until 60-70% confluent and serum-starved for 24 hours for HIT-T15 or 16 hours for MIN6 before 24 h treatment with compounds of the invention. This assay demonstrates that compounds of the invention show an increase in the BrdU incorporation.

Calcium Influx Assay.Measurement of intracellular Ca²⁺ ([Ca²⁺]i) was performed by using a fluorescence laser-imaging plate reader (FLIPR; Molecular Devices). Briefly, MIN6 cells cultured in 384-well plates were incubated for 1 h with 4 μM fluo-3/AM (Molecular Probes) and 0.04% pluronic acid in physiological Locke's buffer (154 mM NaCl, 5.6 mM KCl, 1.0 mM MgCl₂, 2.3 mM CaCl₂, 8.6 mM Hepes, 5.6 mM glucose, and 0.1 mM glycine [pH 7.4]). After incubation, MIN6 cells were washed with Locke's buffer by using an automated cell washer to remove extracellular dye. MIN6 cells were treated with compounds at the indicated concentrations. These cells were excited at 488 nm and Ca²⁺-bound fluo-3 emission was recorded at 500-560 nm.

Real time PCR and Microanalysis experiments were used to show that certain genes, critical for β-cell proliferation, are increased after treatment with a compound of the invention. Mechanistic studies were used to show that some compounds of the invention activated the Ras pathway. For example, (S)-methyl 2,6-dimethyl-5-nitro-4-(2-(trifluoromethyl)phenyl)-1,4-dihydropyridine-3-carboxylate activates the Ras pathway.

### Microarray Analysis

RNA extraction from R7T1 β-cells treated with compounds at the indicated time points is performed using RNeasy kits (Qiagen). The integrity and concentration of RNA is determined by microfluidic analysis on an Experion instrument (BioRad). Standard Affymetrix single amplification is performed using 5 μg total RNA. Standard Affymetrix protocols are used to process Affymetrix MOE430_{—}2 microarrays (Affymetrix). All CEL file images are processed as a single group using gcRMA.

### Real-Time RT-PCR

Total RNA from different samples is isolated and purified using the RNeasy Mini kit with on-column DNase digestion (Qiagen, Valencia, Calif.). Single-stranded cDNA is synthesized from 2 μg of total RNA with the High Capacity cDNA Reverse Transcription Kit (Applied Biosystems). qRT-PCR is performed with the TaqMan® Universal PCR Master Mix, No AmpErase® UNG (Applied Biosystems) on an ABI PRISM 7700 Sequence Detection System. Mouse beta-actin is used for endogenous control. Data are represented as mean±SD of three independent experiments.

### Ras Activation Analysis

Ras-GTP levels are detected using the Active Ras Pull-Down and Detection Kit, following the manufacturer's instructions (Pierce). Briefly, growth-arrested R7T1 β-cells are treated with 0.5 μM of a compound of the invention for an indicated time. Cells are then washed twice with ice-cold phosphate-buffered saline (PBS) and lysed in Lysis/Binding/Wash Buffer containing protease inhibitor cocktail [Roche]. Lysates (500 μg) are clarified by centrifugation and supernatants are incubated with 80 μg of GST-Raf1-RBD fusion protein precoupled to glutathione-Sepharose beads. After incubation for 1 hour at 4° C., beads are washed three times in Lysis/Binding/Wash Buffer, resuspended in Laemmli buffer and boiled. Samples are analyzed by SDS-PAGE (4-20%) followed by transfer to nitrocellulose membrane and then are probed with anti-Ras antibody. Blots are developed using the enhanced ECL system.

### Results and Discussion

High-Throughput Chemical Screen in Immortalized β Cells

To identify small molecules that can induce controlled β-cell proliferation, the reversibly immortalized mouse β-cell line R7T1, which provides the large quantities of homogeneous, functional β cells required for large-scale cell-based screens, was used. SV40 T antigen (TAg) oncoprotein under the control of the Tet-On system was used to immortalize mouse β cells so that these engineered β cells proliferate when TAg is induced in the presence of tetracycline (Tet), but undergo growth arrest upon withdrawal of Tet (see, Efrat S, et al., Proc. Natl. Acad. Sci. USA 92:3576-3580 (1995); Fleischer N, et al, Diabetes 47:1419-1425 (1998); and Milo-Landesman D, et al., Cell Transplant. 10:645-650 (2001)). Although not a true mimic of in vivo quiescent β cells, this system should allow us to identify small molecules that are able to induce reentry into cell cycle and proliferation of growth-arrested cells. Indeed, these engineered cells express high levels of β-cell signature markers, including insulin 1, insulin 2, and Pdx1, and are able to secrete insulin and restore and maintain euglycemia in STZ-treated diabetic mice when induced to growth arrest and transplanted (see, Efrat S, et al., supra; and Milo-Landesman D, et al., supra). A total of ≈850,000 compounds were screened with an automated high-throughput screening platform for their ability to induce the proliferation of growth-arrested β cells in either a 384- or 1,536-well plate format. In the primary screen, proliferation was assayed by using a luminescent assay measuring intracellular ATP content, which is directly proportional to viable cell number (see, Materials and Methods). To rule out any possible Tet mimetics that induce the proliferation of β cells through the induction of SV40 TAg expression, the Tet-Off immortalized β-cell line βTC-Tet was used for a counterscreen. In this system, βTC-Tet cells proliferate in the absence of Tet, but stop dividing in its presence, hence, a Tet analog will not induce the proliferation of growth-arrested βTC-tet cells (see, (see, Efrat S, et al., supra; and Fleischer N, et al, supra). Approximately 80 compounds, representing 10 distinct scaffolds, passed this secondary filter, and were subsequently shown to induce the proliferation of growth-arrested immortalized mouse β cells by direct cell counting.

### Identification of Novel Wnt Agonists as Inducers of β-Cell Proliferation.

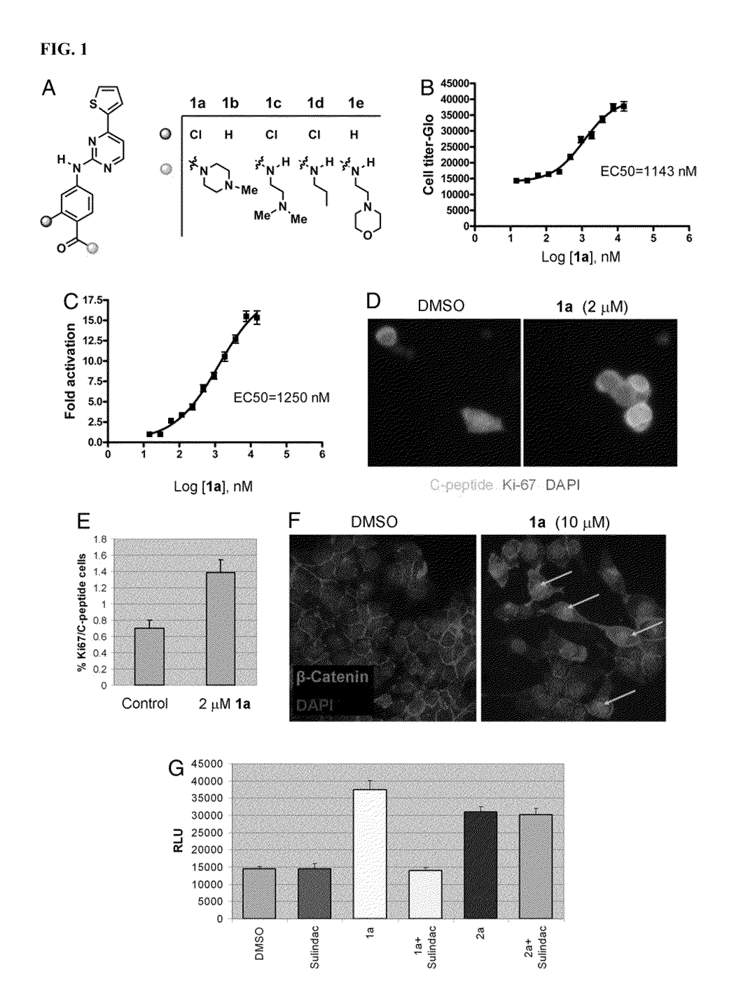

Of the structurally diverse hit compounds, a number have known mechanisms of action. For example, phorbol esters were identified that likely promote β-cell proliferation by binding and activating protein kinase C (PKC) isozymes. Another mechanism by which small molecules might promote β-cell proliferation involves activation of developmental signaling pathways, such as hedgehog and Wnt, which are critical for cellular differentiation and proliferation during embryonic development and adult homeostasis. Indeed, the Wnt pathway has recently been implicated in the regulation of β-cell proliferation based on the phenotypes of mouse models with gain and loss of function in the Wnt signaling pathway (see, Rulifson I C, et al. Proc. Natl. Acad. Sci. USA 104:6247-6252 (2007)). Fifteen compounds, including a group of thiophene-pyrimidines, aminopyridines, and indirubin (BIO), induced the Wnt reporter Super (8×) TOPFlash (see, DasGupta R, et al., Science 308:826-833 (2005)) 5- to 50-fold (FIG. 1A, and data not shown). A representative compound in this group, the thiophene pyrimidine derivative 1a, was further tested for its effects on Wnt signaling and on β-cell proliferation. Compound 1a induced replication of growth-arrested R7T1 β cells in a dose-dependent manner with EC50s of ≈1.1 μM (FIG. 1B). Compound 1a was also able to promote the proliferation of 2 other β-cell lines, HIT-T15 and MIN6. This compound (2 μM) also increased the number of proliferating primary rat β cells staining doubly positive for Ki-67/C-peptide ≈2-fold after incubation for 72 h compared with DMSO treatment (FIGS. 1D and E). Consistent with activation of Wnt signaling, 1a induced the Super (8×) TOPFlash reporter in a dose-dependent manner with an EC50 value of ≈1.25 μM, similar to that for β-cell proliferation (FIG. 1C); at a concentration of 10 μM, 1a activated the Wnt reporter 15-fold compared with that for DMSO. In contrast, compound 1a had no activity on a Wnt reporter (see, DasGupta R, et al., supra) whose β-catenin/TCF binding sites are mutated (data not shown). Increased nuclear β-catenin accumulation was also observed in compound 1a-treated cells (in DMSO-treated control cells, β-catenin staining is weak and appears to concentrate at the cell surface; FIG. 1F). In addition, compound 1a-stimulated Wnt activation and β-cell proliferation were abolished by Sulindac, a Wnt signaling antagonist (FIG. 1G). A preliminary investigation of the mechanism of action of compound 1a revealed that it is a relatively selective and potent (IC50=64 nM) GSK3-β inhibitor, a negative regulator of Wnt signaling pathway (7 of 65 kinases profiled were inhibited by >75% at 5 μM; data not shown). Collectively, these findings suggest that compound 1a likely mediates β-cell proliferation through the Wnt signaling pathway, although the possibility that other signaling pathways are also involved cannot be completely ruled out.

### Agonists of L-Type Calcium Channels Promote β-Cell Proliferation

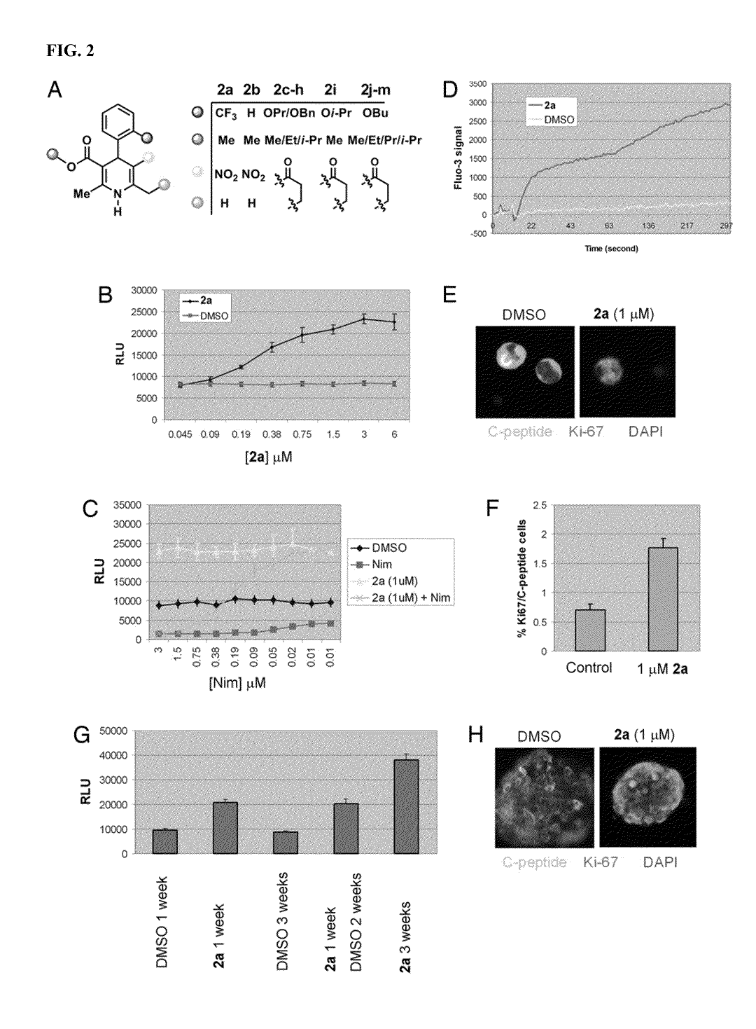

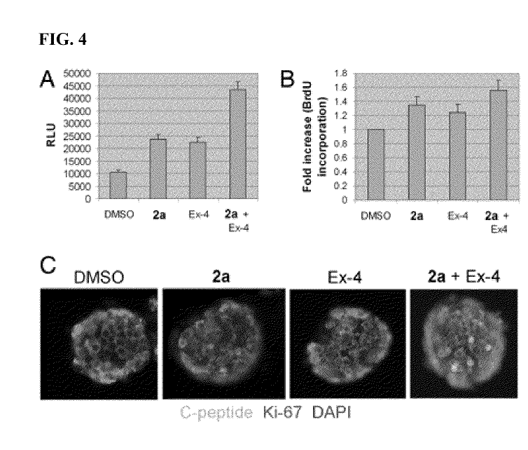

Another group of compounds identified in this screen are dihydropyridine (DHP) derivatives (FIG. 2A), known agonists and antagonists of LTCCs. That the LTCC antagonist nimodipine and agonist Bay K 8644 (2a) induced β-cell death and β-cell proliferation, respectively (FIGS. 2B and C), suggests that the calcium channel agonist activity is responsible for the proliferation of the growth-arrested β cells. Consistent with this notion, the proliferative effect of DHP derivative 2a on β cells can be blocked by nimodipine in a dose-dependent fashion (FIG. 2C), and treatment with 2a leads to a 10-fold increase in transient intracellular calcium levels (FIG. 2D). The compound also increased proliferation of 2 other β-cell lines (HIT-T15 and MIN6) in a dose-dependent manner. Moreover, compound 2a promotes the proliferation of primary rat β cells 2.5-fold as determined by the number of Ki-67 and C-peptide double-positive cells after compound incubation for 3 days (FIGS. 2E and F). Also evaluated whether the proliferative effects of 2a on growth-arrested β cells is strictly dependent on its presence. R7T 0 cells proliferated continuously for up to 3 weeks in the presence of 2a, but stopped dividing upon its removal (FIG. 2G), suggesting that controlled proliferation of β cells by 2a may be possible. A number of experiments indicate that the proliferative effect of 2a is because of the expansion of the β cells per se rather than a dedifferentiated cell type with higher proliferation potential from β cells. First, the expression of β-cell signature markers such as insulin 1, insulin 2, and Pdx1 is unchanged on R7T1 β cells both pretreatment and posttreatment with 2a in gene profiling analysis (data not shown). Second, even after being cultured with 2a for 1-2 weeks, R7T1 cells keep the same morphology and behavior—they remain small and round-shaped and form clusters (data not shown). Moreover, both in cultured primary rat and human islets treated with compound, the overwhelming majority of cells still stain for C-peptide (FIGS. 2H and 4C). Finally, compound 2a also appears to increase the number of proliferating human β cells (FIG. 2H), although quantitation is difficult because of the heterogeneous nature of the human islets used.

Calcium channel-mediated Ca2+ influx regulates diverse cellular processes from muscle contraction and synaptic neurotransmission to cell survival and apoptosis. As a consequence, mutations that affect calcium channel function result in clinical pathologies, referred to as calcium channelopathies. In β cells, calcium channels play a key role in controlling glucose-stimulated insulin secretion and insulin production (see, Yang S. N., et al., Endocr. Rev. 27:621-676 (2006)). Polymorphisms in some calcium channel-encoding genes have been shown to be associated with types 1 and 2 diabetes in genome-wide association studies (see, Yamada Y, et al., Diabetes Metab. Res. Rev. 17:213-216 (2001); Muller Y L, et al. Diabetes 56:3089-3094 (2007); and Sellick G S, et al., Diabetes 52:2636-2638 (2003)). However, it is also possible that these mutations lead to proliferative defects in β cells. In fact, the LTCC α1 D subunit knockout displayed hypoinsulinemia and impaired glucose tolerance in adult mice along with a significant reduction of postnatal β-cell proliferation (see, Namkung Y, et al., J. Clin. Invest. 108:1015-1022 (2001)), suggesting that calcium channel signaling is necessary for β-cell replication. The identification of LTCC agonists as inducers of β-cell proliferation supports the notion that calcium channel signaling modulates β-cell proliferation. Intriguingly, LTCC agonists have also been shown to induce in vivo neurogenesis (see, Deisseroth K, et al., Neuron 42:535-552 (2004)), and more recently, isoxazole derivatives were reported to trigger neuronal differentiation in uncommitted adult neural stem cells and to increase the proliferation of committed neuroblasts by activating Ca²⁺ influx through both voltage-gated Ca²⁺ channels and N-methyl-d-aspartic acid (NMDA) receptors (see, Schneider J W, et al., Nat. Chem. Biol. 4:408-410 (2008)).

### Mechanistic Studies of LTCC Agonists

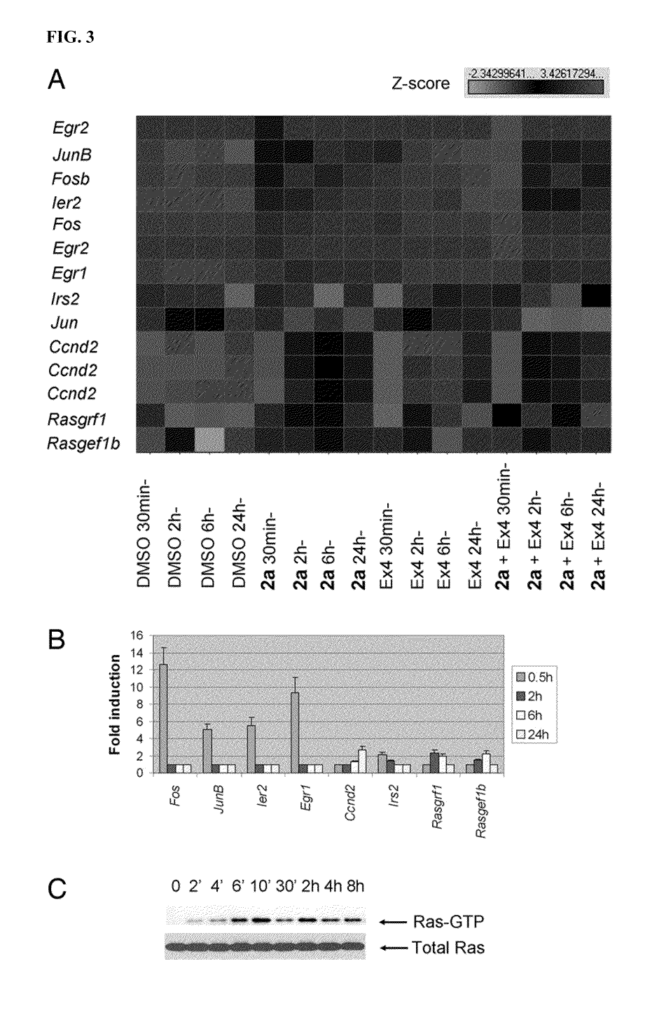

To further explore the mechanism of action of 2a, its effects on gene expression in growth-arrested R7T1 β cells were analyzed by microarray analysis. Treatment of cells with 0.5 μM 2a led to a large increase in the expression of several immediate-early genes, including Fos, Jun, and Egr families, which are critical for the expression of genes important for cell survival and cell cycle progression, within 30 min (FIG. 3A). In addition, expression of Ccnd2 (cyclin D2), an important positive regulator during G1 phase, increased approximately 1.7-fold after treatment with 2a for 24 h, and IRS2, which is critical for β-cell survival and proliferation, was up-regulated 1.7-fold (FIG. 3A). The expression profiles of these genes were confirmed by quantitative real-time RT-PCR (FIG. 3B). Calcium signaling has been shown to be transduced by the activation of transcription factors such as CREB and NFAT (see, Im S H, et al., Mol Cells 18:1-9 (2004); and Screaton R A, et al., Cell 119:61-74 (2004)). In neurons, the effects of calcium signaling are mediated in part by the small GTPase Ras/MAPK pathway, based on the observation that Ras signaling is activated upon treatment with a high concentration of potassium chloride, which increases calcium influx by depolarizing the cell membrane of neurons (see, Farnsworth C L, et al., Nature 376:524-527 (1995); and Dolmetsch R E, et al., Science 294:333-339 (2001)). Further investigated was whether the LTCC agonist 2a is able to directly activate Ras signaling. Indeed, the level of Ras-GTP, the active Ras form, was elevated after treatment of growth-arrested R7T cells with 2a for 6 min (FIG. 3C). Ras is activated and deactivated by the recruitment of guanine nucleotide exchange factors (GEFs) and GTPase activating proteins (GAPs) to the membrane, respectively. Calcium signaling modulates Ras signaling by regulating the rapid recruitment of GEFs and GAPs to the plasma membrane (see, Cook S J, et al., Cell Calcium 39:101-112 (2006)). Strikingly, the microarray data showed that 2 GEFs (RasGRF1 and RasGEF1b) were transcriptionally up-regulated in growth-arrested R7T1 β cells upon treatment with 2a for 2-6 h (FIG. 3A). These findings were further validated by quantitative RT-PCR analysis (FIG. 3B). Consistent with these findings, Ras-GTP levels were elevated for an extended time after the peak level at 10 min of incubation (FIG. 3C). These findings suggest that LTCC agonists likely activate Ras signaling through both short- and longer-term mechanisms by the recruitment of GEFs to the plasma membrane and their enhanced expression, respectively. A sustained Ras activity is required for cell cycle initiation and progression up to the restriction point in the late G1 phase (see, Coleman M L, et al., Nat. Rev. Mol. Cell. Biol. 5:355-366 (2004)). How this transcriptional regulation of GEFs is mediated by LTCC-mediated calcium signaling needs further investigation (FIG. 3A).

### Additive Effect of LTCC Agonist 2a and Glp-1 on β-Cell Replication

Finally, further examined was whether Ca²⁺- and cAMP-dependent pathways might function in concert to promote β-cell proliferation. The gut hormone glucagon-like peptide-1 (GLP-1) promotes β-cell proliferation and viability by increasing cAMP concentration (see, Brubaker P L, et al., Endocrinology 145:2653-2659 (2004)), which is in part mediated by activation of the cAMP- and calcium-responsive transcription factor CREB (see, Jhala U S, et al., Genes Dev. 17:1575-1580 (2003)). It has also been reported that cAMP and calcium pathways converge on CREB to regulate β-cell gene expression in response to glucose and incretin hormones (see, Screaton R A, et al., supra). Thus, 2a and GLP-1 might be expected to exert an additive effect on β-cell proliferation. Indeed, treatment of growth-arrested R7T1 cells, HIT-T15 cells, and primary human β cells with 1 μM 2a and 5 nM of the GLP-1 analog Ex-4 led to a higher rate of proliferation than either alone (FIG. 4A-C). These data suggest that cAMP and calcium pathways function in a concerted manner in promoting β-cell proliferation. Consistent with these findings, several early-response genes, such as Fos, FosB, JunB, Egr1, Egr2, and Ier2, were expressed at higher levels in cells treated with both 2a and Ex-4 than with either alone (FIG. 3A). Moreover, though increased Ccnd2 expression is evident by 24 h after treatment with 2a, its expression increases much earlier (at 2 h) and to a higher level at 24 h in 2a and Ex-4 co-treated cells (FIG. 3A). Similarly, combined treatment of growth-arrested R7T1 cells with 2a and Ex-4 also triggers higher expression of 2 RasGEFs, Rasgrf1, and Rasgef1b, which may contribute to the additive effect of 2a and Ex-4.

In view of the foregoing, it is clear that the present invention provides compounds that induce pancreatic β-cell proliferation. One group of compounds induces β-cell proliferation by activating Wnt signaling, which is consistent with a recent report that GSK30 inhibitors promote β-cell survival and replication (see, Mussmann R, et al., J. Biol. Chem. 282:12030-12037 (2007)). L-type calcium channel agonists were also identified as inducers of (β-cell proliferation/regeneration. It is thought that this class of compounds, which includes, e.g., the compounds of Formula Ie, offer a potentially promising avenue for treatment of β-cell deficiency in diabetes, especially when combined with GLP-1 receptor agonists.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

What is claimed is:

1. A compound selected from Formula Ia: 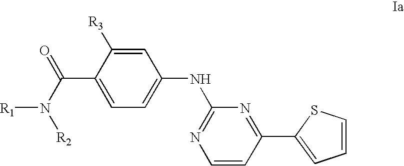
wherein:
R₁ is selected from hydrogen and C₁₋₆alkyl;
R₂ is selected from C₁₋₆alkyl and —X₁NR₄R₅; wherein X₁ is C₁₋₄alkylene; R₄ and R₅ are independently selected from hydrogen and C₁₋₄alkyl; or R₄ and R₅ together with the nitrogen to which they are both attached, and optionally with another heteroatom chosen from the group O, S and N, form a 6 member heterocycle containing 1 to 2 heteroatoms; or R₁ and R₂ together with the nitrogen to which they are both attached, and optionally with another heteroatom chosen from the group O, S and N, form a 6 member heterocycle containing 1 to 2 heteroatoms; wherein said heterocycle formed from R₁ and R₂ or R₄ and R₅ can be optionally substituted with C₁₋₄alkyl; and
R₃ is selected from hydrogen, halo, C₁₋₄alkyl, halo-substituted-C₁₋₄alkyl, C₁₋₄alkoxy and halo-substituted-C₁₋₄alkoxy.

2. The compound of claim 1 in which: R₁ is hydrogen; R₂ is selected from dimethyl-amino-ethyl, propyl and morpholino-ethyl; or R₁ and R₂ together with the nitrogen to which they are both attached form piperazinyl substituted with methyl.

3. The compound of claim 2 selected from: (2-chloro-4-(4-(thiophen-2-yl)pyrimidin-2-ylamino)phenyl)(4-methylpiperazin-1-yl)methanone; (4-methylpiperazin-1-yl)(4-(4-(thiophen-2-yl)pyrimidin-2-ylamino)phenyl)methanone; 2-chloro-N-(2-(dimethylamino)ethyl)-4-(4-(thiophen-2-yl)pyrimidin-2-ylamino)benzamide; 2-chloro-N-propyl-4-(4-(thiophen-2-yl)pyrimidin-2-ylamino)benzamide; and 2-chloro-N-(2-morpholinoethyl)-4-(4-(thiophen-2-yl)pyrimidin-2-ylamino)benzamide.

4. A compound selected from Formula Ib and Ic: 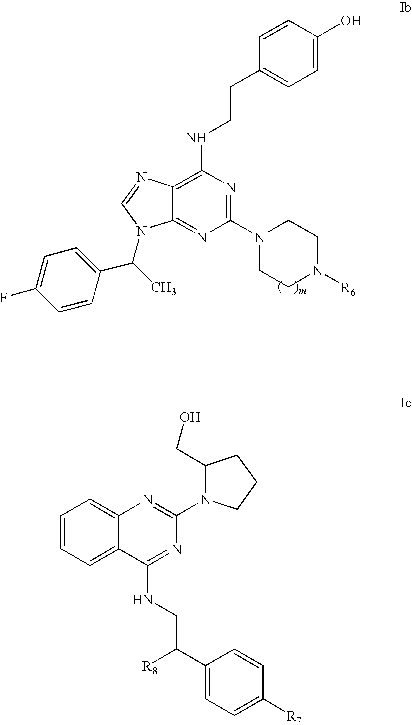
wherein in Formula Ib:
m is selected from 0, 1 and 2; and
R₆ is C₁₋₆alkyl; and
wherein in Formula Ic:
R₇ is selected from hydrogen, halo, C₁₋₄alkyl and C₁₋₄alkoxy; and
R₈ is selected from hydrogen, C₁₋₄alkyl and C₆₋₁₀aryl.

5. The compound of claim 4, wherein in Formula Ib:
m is selected from 1 and 2; and
R₆ is selected from methyl and ethyl; and
wherein Formula Ic:
R₇ is selected from hydrogen, bromo, methyl and methoxy; and
R₈ is selected from hydrogen and phenyl.

6. The compound of claim 5 selected from: 4-(2-(2-(4-ethylpiperazin-1-yl)-9-(1-(4-fluorophenyl)ethyl)-9H-purin-6-yl-amino)ethyl)phenol; 4-(2-(9-(1-(4-fluorophenyl)ethyl)-2-(4-methylpiperazin-1-yl)-9H-purin-6-ylamino)ethyl)phenol; 4-(2-(9-(1-(4-fluorophenyl)ethyl)-2-(4-methyl-1,4-diazepan-1-yl)-9H-purin-6-ylamino)ethyl)phenol; (1-(4-(4-methoxyphenethylamino)quinazolin-2-yl)pyrrolidin-2-yl)methanol; (1-(4-(4-bromophenethylamino)quinazolin-2-yl)pyrrolidin-2-yl)methanol; (1-(4-(4-methylphenethylamino)quinazolin-2-yl)pyrrolidin-2-yl)methanol; and (1-(4-(2,2-diphenylethylamino)quinazolin-2-yl)pyrrolidin-2-yl)methanol.

7. A method of increasing the number of pancreatic β-cells, said method comprising contacting the pancreatic β-cells with a compound of claim 1 or 4.

8. The method of claim 7, wherein the method is carried out in vivo, in vitro or ex vivo.

9. The method of claim 8, wherein the pancreatic β-cells are human.

10. A method of treating a disease or disorder resulting from the reduced activity or destruction of pancreatic β-cells comprising administering to a patient in need of such treatment pancreatic β-cells expanded by a compound of claim 1 or 4.

11. The method of claim 10, wherein the disease or disorder is selected from type 1 and type 2 diabetes.

12. A method of using a compound of Formula Id to increase the number of pancreatic β-cells, said method comprising contacting the pancreatic β-cells with a compound of Formula Id: 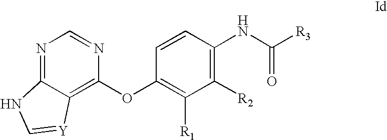
wherein:
Y is selected from N and CH;
R₁ is selected from hydrogen and C₁₋₆alkyl;
R₂ is selected from hydrogen and C₁₋₆alkyl; or R₁ and R₂, together with the fragment of the phenyl ring to which R₁ and R₂ are attached, and optionally with a heteroatom chosen from the group O, S and N, form a C₆₋₁₀aryl or C₅₋₁₀heteroaryl;
R₃ is selected from NR₄R₅ and X₁R₅; wherein X₁ is selected from a bond and C₁₋₄alkylene; R₄ is selected from hydrogen and C₁₋₆alkyl; R₅ is selected from C₆₋₁₀aryl optionally substituted with 1 to 3 radicals independently selected from halo-substituted-C₁₋₆alkyl, C₁₋₆alkoxy, C₅₋₁₀heteroaryl, C₃₋₈heterocycloalkyl, C₅₋₁₀heteroaryl-C₁₋₄alkyl and C₃₋₈heterocycloalkyl-C₁₋₄alkyl; wherein said heteroaryl and heterocycloalkyl substituents of R₅ are optionally substituted with C₁₋₆alkyl; and the pharmaceutically acceptable salts, hydrates, solvates and isomers thereof.

13. The method of claim 12, wherein R₁ and R₂ are both hydrogen or R₁ and R₂, together with the fragment of the phenyl ring to which R₁ and R₂ are attached, and optionally with a heteroatom chosen from the group O, S and N, form quinolinyl or naphthalenyl.

14. The method of claim 13, wherein R₃ is selected from NHR₅ and X₁R₅; wherein X₁ is selected from a bond and methylene; R₅ is selected from phenyl optionally substituted with 1 to 3 radicals independently selected from trifluoro-methyl, methoxy, imidazolyl and piperazinyl-methyl; wherein said imidazolyl or piperazinyl substituents of R₅ are optionally substituted with methyl and ethyl.

15. The method of claim 14, wherein the compound of Formula Id is selected from: 1-[4-(7H-Pyrrolo[2,3-d]pyrimidin-4-yloxy)-phenyl]-3-(3-trifluoromethyl-phenyl)-urea; 1-[4-(4-Ethyl-piperazin-1-ylmethyl)-3-trifluoromethyl-phenyl]-3-[4-(7H-pyrrolo[2,3-d]pyrimidin-4-yloxy)-phenyl]-urea; 1-[3-(4-Methyl-imidazol-1-yl)-5-trifluoromethyl-phenyl]-3-[4-(7H-pyrrolo[2,3-d]pyrimidin-4-yloxy)-phenyl]-urea; 1-(3,5-Dimethoxy-phenyl)-3-[4-(7H-pyrrolo[2,3-d]pyrimidin-4-yloxy)-phenyl]-urea; 1-[4-(9H-Purin-6-yloxy)-phenyl]-3-(3-trifluoromethyl-phenyl)-urea; 1-[3-(4-M ethyl-imidazol-1-yl)-5-trifluoromethyl-phenyl]-3-[4-(9H-purin-6-yloxy)-phenyl]-urea; 1-[4-(4-Ethyl-piperazin-1-ylmethyl)-3-trifluoromethyl-phenyl]-3-[4-(9H-purin-6-yloxy)-phenyl]-urea; 1-[5-(7H-Pyrrolo[2,3-d]pyrimidin-4-yloxy)-quinolin-8-yl]-3-(3-trifluoromethyl-phenyl)-urea; N-[4-(7H-Pyrrolo[2,3-d]pyrimidin-4-yloxy)-phenyl]-2-(3-trifluoromethyl-phenyl)-acetamide; 2-[3-(4-M ethyl-imidazol-1-yl)-5-trifluoromethyl-phenyl]-N-[4-(9H-purin-6-yloxy)-phenyl]-acetamide; N-[4-(7H-Pyrrolo[2,3-d]pyrimidin-4-yloxy)-phenyl]-3-trifluoromethyl-benzamide; 3-(4-Methyl-imidazol-1-yl)-N-[4-(7H-pyrrolo[2,3-d]pyrimidin-4-yloxy)-phenyl]-5-trifluoromethyl-benzamide; N-[4-(9H-Purin-6-yloxy)-phenyl]-3-trifluoromethyl-benzamide; 4-(4-Ethyl-piperazin-1-ylmethyl)-N-[4-(9H-purin-6-yloxy)-phenyl]-3-trifluoromethyl-benzamide; and N-[5-(9H-Purin-6-yloxy)-quinolin-8-yl]-3-trifluoromethyl-benzamide.

16. The method of claim 15, wherein the method is carried out in vivo, in vitro or ex vivo.

17. The method of claim 16, wherein the pancreatic β-cells are human.

18. The method of claim 17, wherein the pancreatic β-cells are derived from a human donor.

19. A method of treating a disease or disorder resulting from the reduced activity or destruction of pancreatic β-cells comprising administering to a patient in need of such treatment pancreatic β-cells expanded by a compound of Formula Id: 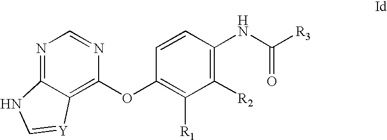
wherein:
Y is selected from N and CH;
R₁ is selected from hydrogen and C₁₋₆alkyl;
R₂ is selected from hydrogen and C₁₋₆alkyl; or R₁ and R₂, together with the fragment of the phenyl ring to which R₁ and R₂ are attached, and optionally with a heteroatom chosen from the group O, S and N, form a C₆₋₁₀aryl or C₅₋₁₀heteroaryl;
R₃ is selected from NR₄R₅ and X₁R₅; wherein X₁ is selected from a bond and C₁₋₄alkylene; R₄ is selected from hydrogen and C₁₋₆alkyl; R₅ is selected from C₆₋₁₀aryl optionally substituted with 1 to 3 radicals independently selected from halo-substituted-C₁₋₆alkyl, C₁₋₆alkoxy, C₅₋₁₀heteroaryl, C₃₋₈heterocycloalkyl, C₅₋₁₀heteroaryl-C₁₋₄alkyl and C₃₋₈heterocycloalkyl-C₁₋₄alkyl; wherein said heteroaryl and heterocycloalkyl substituents of R₅ are optionally substituted with C₁₋₆alkyl; and the pharmaceutically acceptable salts, hydrates, solvates and isomers thereof.

20. The method of claim 19, wherein the disease or disorder is selected from type 1 and type 2 diabetes.

21. A method of using a compound of Formula Ie to increase the number of pancreatic β-cells, said method comprising contacting the pancreatic β-cells with a compound of Formula Ie: 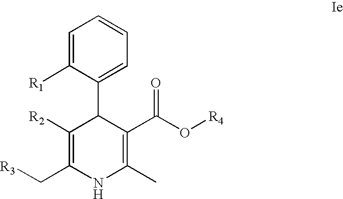
wherein:
R₁ is selected from hydrogen, halo, C₁₋₄alkyl, C₁₋₄alkoxy, halo-substituted-C₁₋₄alkyl, halo-substituted-C₁₋₄alkoxy, phenoxy and benzyloxy;
R₂ is selected from nitro and —C(O)R₅; wherein R₅ is selected from hydrogen and C₁₋₄alkyl;
R₃ is selected from hydrogen and C₁₋₄alkyl; or R₂ and R₃ together with the carbon atoms to which R₂ and R₃ are attached form a partially saturated 4 to 7 member cyclic group fused to the 1,4-dihydropyridine core of Formula Ie; and
R₄ is selected from hydrogen and C₁₋₄alkyl.

22. The method of claim 21, wherein R₁ is selected from hydrogen, trifluoromethyl, propoxy, phenoxy, benzyloxy, butoxy and isopropoxy; R₂ is from nitro; R₃ is selected from hydrogen and C₁₋₄alkyl; or R₂ and R₃ together with the carbon atoms to which R₂ and R₃ are attached form a partially saturated 4 to 7 member cyclic group fused to the 1,4-dihydropyridine core of Formula Ie; and R₄ is selected from methyl, ethyl, propyl and isopropyl.

23. The method of claim 22, wherein the compound of Formula Ie is selected from: (S)-methyl 2,6-dimethyl-5-nitro-4-(2-(trifluoromethyl)phenyl)-1,4-dihydropyridine-3-carboxylate; (S)-methyl 2,6-dimethyl-5-nitro-4-phenyl-1,4-dihydropyridine-3-carboxylate; (R)-methyl 2-methyl-5-oxo-4-(2-propoxyphenyl)-1,4,5,6,7,8-hexahydroquinoline-3-carboxylate; (R)-ethyl 2-methyl-5-oxo-4-(2-propoxyphenyl)-1,4,5,6,7,8-hexahydroquinoline-3-carboxylate; (R)-isopropyl 2-methyl-5-oxo-4-(2-propoxyphenyl)-1,4,5,6,7,8-hexahydroquinoline-3-carboxylate; (R)-isopropyl 4-(2-(benzyloxy)phenyl)-2-methyl-5-oxo-1,4,5,6,7,8-hexahydroquinoline-3-carboxylate; (R)-ethyl 4-(2-(benzyloxy)phenyl)-2-methyl-5-oxo-1,4,5,6,7,8-hexahydroquinoline-3-carboxylate; (R)-methyl 4-(2-(benzyloxy)phenyl)-2-methyl-5-oxo-1,4,5,6,7,8-hexahydroquinoline-3-carboxylate; (R)-methyl 4-(2-isopropoxyphenyl)-2-methyl-5-oxo-1,4,5,6,7,8-hexahydroquinoline-3-carboxylate; (R)-methyl 4-(2-butoxyphenyl)-2-methyl-5-oxo-1,4,5,6,7,8-hexahydroquinoline-3-carboxylate; (R)-isopropyl 4-(2-butoxyphenyl)-2-methyl-5-oxo-1,4,5,6,7,8-hexahydroquinoline-3-carboxylate; (R)-ethyl 4-(2-butoxyphenyl)-2-methyl-5-oxo-1,4,5,6,7,8-hexahydroquinoline-3-carboxylate; (R)-propyl 4-(2-butoxyphenyl)-2-methyl-5-oxo-1,4,5,6,7,8-hexahydroquinoline-3-carboxylate; and (R)-methyl 4-(2-butoxyphenyl)-2-methyl-5-oxo-1,4,5,6,7,8-hexahydroquinoline-3-carboxylate.

24. The method of claim 23, wherein the method is carried out in vivo, in vitro or ex vivo.

25. The method of claim 24, wherein the pancreatic β-cells are human.

26. The method of claim 25, wherein the pancreatic β-cells are derived from a human donor.

27. The method of claim 21, further comprising contacting the pancreatic β-cells with a GLP-1 receptor agonist.

28. A method of treating a disease or disorder resulting from the reduced activity or destruction of pancreatic β-cells comprising administering to a patient in need of such treatment pancreatic β-cells expanded by a compound of Formula Ie.

29. The method of claim 28, wherein the disease or disorder is selected from type 1 and type 2 diabetes.

30. The method of claim 28, further comprising administering to said patient a GLP-1 receptor agonist.
